(19) United States
(12) Patent Application Publication
(10) Pub. No.: US 2019/0091307 A1
(43) Pub. Date: Mar. 28, 2019

(54) PTPS-BASED VACCINES AGAINST CANCER

(71) Applicant: INSTITUT GUSTAVE ROUSSY, VILLEJUIF (FR)

(72) Inventors: SEBASTIEN APCHER, FRANCONVILLE (FR); ROBIN FAHRAEUS, PARIS (FR); TAKAHIRO YAMAZAKI, VILLEJUIF (FR); ALISON PIERSON, VILLEJUIF (FR); MATHILDE BOULPICANTE, CHEVILLY-LARUE (FR)

(21) Appl. No.: 16/081,073

(22) PCT Filed: Mar. 3, 2017
(86) PCT No.: PCT/EP2017/055004

§ 371 Date: Aug. 30, 2018

(30) Foreign Application Priority Data
Mar. 3, 2016 (EP) 16305243.4

(51) Int. Cl. A61K 39/00; A61P 35/00

(52) U.S. Cl. CPC A61K 39/0011; A61K 39/00119; A61K 2039/53; A61K 2039/5152; A61P 35/00

(57) ABSTRACT

The present invention relates to the field of medicine. It more particularly relates to peptides, microvesicles containing such peptides, compositions containing same, in particular vaccine, and methods for stimulating an immune response in a subject.

### FIELD OF THE INVENTION

The present invention relates to the field of medicine and is typically used in therapeutic and prophylactic areas. The invention more particularly relates to a Pioneer Translation Product (“PTP”) consisting in a peptide having 7 to 50 amino acids, to microvesicles containing such a PTP, to compositions containing same, in particular vaccine compositions, and to methods for stimulating an immune response in a subject, preferably directed against a tumor antigen.

### BACKGROUND OF THE INVENTION

The main goal of vaccination is to induce an effective immune response that can control viral infectious diseases and cancer in humans. The immune system is classified into two categories: on one hand the innate immune system and on the other hand the adaptive immune system. Cellular immune reactions against infected or transformed cells require the activation of the adaptive immune system. This activation can be achieved only by stimulating antigen-specific cytotoxic T lymphocytes such as CD8⁺ T cells, B cells and T helper T cells like CD4⁺ T cells. In fact cytotoxic CD8⁺ T cells are able to detect viral infected cells or cancerous cells that present on their cell surface antigens that are bound to MHC class I molecules. This recognition has for consequence a direct cytotoxic action of the T cells towards the infected cells or the tumor cells. Nevertheless, the proper immune reaction against these different states requires the activation of CD8⁺ T cells by professional antigen presenting cells (pAPCs), such as dendritic cells and macrophages, which take up external peptide material to present them on their MHC class I molecules through a process called cross-presentation. The direct and cross-presentation pathways are fundamental processes for the detection and elimination of cells that pose a threat to the host. This process is further dependent on T helper T cells that recognized antigen in the form of short peptides of 13-20 amino acids derived from exogenous proteins bound to MHC class II molecules.

Some years ago, it was postulated that the source of peptides for direct presentation to the MHC class I restricted pathway is not derived from the degradation of full length proteins but from so-called defective ribosomal products, or DRiPs. Further studies have since supported this notion, even though the actual source of peptides for the class I pathway was not known. Inventors have shown that the latent protein EBNA1 of the Epstein barr virus affects mRNA translation in order to suppress antigenic presentation and, in that way, avoids its detection. Moreover, they have observed that the rate of mRNA translation is closely related to antigen presentation. In addition, some MHC class I-bound peptides have been described as being generated from cryptic translation, which refers to polypeptides synthesized in the cell from non-conventional translational mechanisms. These can either be peptides encoded by intron, intron/exon junctions, 5′ and 3′ untranslated regions or alternate translational reading frame. All these observations led to a shift of focus from protein degradation to mRNA translation as being the critical process for antigenic production. More recently, inventors have shown that antigenic presentation is equivalent whether peptide is expressed intronically vs. exonically and give rise to the so called Pioneer Translation Products (PTPs), which are produced by a translation event distinct from the canonical event giving rise to full length proteins. The previous results were supported by the fact that if mRNA exports, from the nucleus to the cytoplasm, was blocked, the antigenic presentation was markedly enhanced from exon and intron-encoded peptides. Overall, inventors have demonstrated that antigenic peptides for the MHC class I pathway are to a large extent derived from an mRNA translation event that is different and independent from that producing full length proteins and that takes place during the early scanning of newly synthesized mRNAs in the nuclear compartment (Apcher, Millot et al. 2013, Apcher, Daskalogianni et al. 2015). These PTPs are likely to constitute the elusive DRiPs. They can be generated before mRNA splicing occurs which, for example, offers an explanation to how the immune system can “tolerate” tissue-dependent alternative splicing products.

Nowadays, therapeutic vaccination in cancer immunotherapy aiming at improving the host immune mediated tumor recognition and destruction is experiencing renewed enthusiasm. But in 1996, a class I binding synthetic epitope derived from the MAGE-1 protein was already tested as a peptide based vaccine in a clinical trial. Nevertheless the same group and others, using synthetic epitopes as vaccines could not see any beneficial clinical responses in melanoma patients. Then, other short peptides directed against different cancer have been used without again demonstrating any beneficial T cell responses in patients. Then, multiple peptides vaccines have been used especially against melanoma without any breakthroughs. Recently, it has been shown that immunizations with synthetic long peptides (more than 20 amino acids) were more immunogenic and had an anti-tumor growth effect better than immunizations observed with short peptides. These differences between the two kinds of peptides containing minimal short antigenic epitopes may be found in the fact that longer peptides can be protected against extracellular degradations due to their tertiary structures and in the fact that they are too long to bind directly to MHC class I molecules of any cell lines. In addition, the benefit of using longer peptides as vaccine is that they need to be internalized and require appropriate processing by the proteasome in pAPCs before being presented at the cell surface and activate CD8⁺ T cells. Moreover, longer peptides have a better chance of containing several epitopes that may induce activation of different CD8⁺ T cells and so induce multiple immune responses.

Exosomes secreted by immune cells or tumor cells have been investigated for their potential in tumor immunotherapy. Exosomes originate as intralumenal vesicles in the multivesicular body (MVB), and the incorporation of specific proteins is selective. Exosomes are vesicles having a diameter of 30 to 100 nm. It has been hypothesized that tumor derived exosomes could contain tumor antigens and thus be used as a source of tumor antigens for cancer-vaccines. Also, many groups have reported that dendritic cell (DC)-derived exosomes can be useful and effective agents for inducing a specific anti-tumor immunity. Nevertheless increasing lines of evidence suggest that tumor-derived exosomes are imperfect as they can induce tumor immune evasion with different roles in different pathways such as by inhibiting the differentiation of DCs or by negatively regulating the NK cells (Valenti, Huber et al. 2006, Clayton, Mitchell et al. 2008, Whiteside, Mandapathil et al. 2011).

Inventors now herein describe a vaccine composition comprising PTPs, produced from intron or exons sequences, preferably in combination with microvesicles containing PTPs, typically exosomes, which is able to induce an appropriate CD8⁺ T cell immune response against a tumor allowing the complete inhibition of the tumor growth, preferably the tumor destruction.

### SUMMARY OF THE INVENTION

The present invention concerns products and methods for improving antigen specific immune responses, in particular in the field of cancer therapy and prophylaxis. The present invention is based on the unexpected finding that a Pioneer Translation Product (“PTP”) consisting in a peptide having 7 to 50 amino acids, typically comprising at least one MHC class I epitope, preferably comprising at least one MHC class I epitope and at least one MHC class II epitope, is capable of inducing, in a subject suffering of a cancer, an efficient, preferably sustained, immune response against a tumor expressing such a peptide.

A first object of the invention thus relates to a Pioneer Translation Product (“PTP”) consisting in a peptide having 7 to 50 amino acids, typically of 5 kDa or less, for use as vaccine, preferably as a cancer vaccine, in a subject. The PTP is typically expressed from a sequence selected from an intron, a 3′ or 5′ untranslated region (UTR), a LncRNA (Long non coding RNA), a miRNA (microRNA), an intergenic sequence and a combination thereof. The PTP preferably comprises at least one MHC class I epitope and/or at least one MHC class II epitope.

A second object of the invention relates to a microvesicle, typically an exosome or an equivalent tumor-derived microvesicle such as a melanosome, comprising at least one PTP (preferably several PTPs), typically a PTP as herein described, said PTP preferably comprising at least one MHC class I epitope and/or at least one MHC class II epitope.

A third object of the invention relates to a composition, in particular a vaccine composition, comprising at least one PTP (preferably several PTPs) and/or a microvesicle, typically an exosome or a tumor-derived microvesicle as herein described, and a pharmaceutically acceptable carrier or excipient.

A preferred vaccine composition comprises at least a first PTP as herein described, a microvesicle and a pharmaceutically acceptable carrier or excipient. Preferably, the microvesicle comprises at least one second PTP consisting in a peptide having 7 to 50 amino acids, said second PTP preferably comprising at least one MHC class I epitope and/or at least one MHC class II epitope, the microvesicle optionally comprising the first PTP.

The invention also relates to a nucleic acid sequence encoding a PTP for use as a vaccine according to the invention and to a composition, in particular a vaccine composition, comprising such a nucleic acid sequence and a pharmaceutically acceptable carrier or excipient.

In a preferred aspect, the herein described vaccine composition is for use in a human being

The present invention also relates to the use of such a PTP, microvesicle, nucleic acid or composition for preventing or treating cancer in a subject.

Another object of the invention relates to a method of producing an immune response in a subject, or of vaccinating a subject, against a specific antigen, preferably a tumor antigen, the method comprising injecting to said subject a PTP according to the invention derived from said antigen, a microvesicle including said PTP, or a vaccine composition including said PTP.

A further object of the invention relates to a method of preventing or treating cancer in a subject, the method comprising injecting to said subject a PTP according to the invention, preferably a PTP derived from a polypeptide expressed by a cancerous tumor of the subject, a microvesicle including said PTP, or a vaccine composition including said PTP.

### DETAILED DESCRIPTION OF THE INVENTION

The major histocompatibility complex (MHC) class I antigen presentation pathway allows the immune system to distinguish between self and non-self. Despite extensive research on the processing of antigenic peptides, little is known about their origin. Inventors revealed that a unique class of peptides, termed Pioneer Translation Products (“PTPs”), is produced during the pioneer rounds of mRNA translation and provides the major source of antigenic peptide substrates for direct presentation to the MHC class I pathway. They have demonstrated that a major proportion of the substrates for the MHC class I pathway is synthesized during the early steps of mRNA maturation via a noncanonical translation mechanism within the nucleus and before introns are spliced out (Apcher et al., 2013). This mechanism is independent from that producing full length protein. Inventors have also demonstrated that PTPs are a better source of peptides for the MHC class I cross presentation pathway than full length protein and now herein reveal that these PTPs, in particular when combined to microvesicles, can be used as a vaccine, in particular for efficiently preventing or treating cancer.

A first object of the invention thus relates to a Pioneer Translation Product (“PTP”) consisting in a peptide having 7 to 50 amino acid residues or 8 to 50 amino acid residues for use as vaccine, preferably as a cancer vaccine, in a subject. Pioneer Translation Products (“PTPs”) are herein defined as peptides derived from non-spliced mRNA that are expressed from intron, exon, 3′ and 5′ untranslated regions (UTR), LncRNA (Long non coding RNA), miRNA (microRNA) and/or intergenic sequences. Preferably, the PTP consists in a peptide having 7 to 50 amino acids which is expressed from a sequence selected i) from an intron, a 3′ or 5′ untranslated region (UTR), a LncRNA (Long non coding RNA), a miRNA (microRNA), an intergenic sequence and a combination thereof, ii) an intron, a LncRNA (Long non coding RNA), a miRNA (microRNA), an intergenic sequence and a combination thereof or iii) an intron, a LncRNA (Long non coding RNA), a miRNA (microRNA) and an intergenic sequence. PTPs are produced by a translation event distinct from the canonical event giving rise to full length proteins that takes place during the early scanning of newly synthesized mRNAs in the nuclear compartment (Apcher, Millot et al. 2013, Apcher, Daskalogianni et al. 2015). These PTPs are preferably not of viral or bacterial origin. They typically consist in a sequence of 7 to 50 amino acid residues and have an atomic mass of 5 kDa or less, typically 3 kDa or less. A PTP preferably consists in a sequence of 7 to 30 amino acid residues, for example of 7 to 27 amino acid residues. A PTP can for example comprise 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 amino acid residues. A PTP typically comprises a MHC class I epitope. A PTP purified from the nuclear compartment of a tumor cell [also herein identified as “tumor-associated PTP” (TA-PTPs)] typically elicits a specific anti-tumor CD8⁺ T cell response against the tumor from which the tumor cell is derived. A preferred PTP according to the invention comprises at least a MHC class I epitope and/or a MHC class II epitope, the MHC class II epitope eliciting a long lasting CD4⁺ T cell response from the immune system which extends the anti-tumor CD8+ T cell response.

A PTP of the invention can be obtained or purified from (and is said to be “derived from”) any protein, polypeptide or antigen against which a specific immune response is to be elicited in the subject to be treated/vaccinated using standard biochemical approaches. In the context of a tumor, PTP extraction involves the lysis of tumor cells with detergent or salt followed by the extraction of peptides of 5 kDa or less, preferably 3 kDa or less, and purification thereof by standard chromatography approaches including anionic or hydrophobic chromatography and/or affinity chromatography on columns.

In another embodiment of the invention, antigenic epitope derived from PTPs can be eluted from tumor cell surface by citrate phosphate buffer (pH 3.3). The antigenic epitope can be analyzed by mass spectrometry and a peptide de novo sequencing can be done. The analytical process indeed allows the deduction of peptide's amino acid sequence from the tandem mass spectrum (MS/MS) without using a sequence database. After identification of epitopes from intron, exon, 3′ and 5′ UTRs, LncRNA, miRNA or intergenic regions, new PTPs containing different MHC class I and/or class II epitopes can be synthesized.

In a particular embodiment of the invention, the PTP of the invention comprises at least one MHC class I epitope and/or at least one MHC class II epitope. Preferably the PTP of the invention comprises at least one MHC class I epitope and at least one MHC class II epitope.

In a preferred embodiment, the PTP is a PTP activating CD4⁺ T cells and/or CD8⁺ T cells.

A particular PTP herein described is a PTP selected from anyone of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22 and SEQ ID NO: 23 (cf. Tables 1 and 2 and sequence listing).

In a preferred embodiment, the PTP for use as a cancer vaccine in a subject is a PTP derived from the cancer tumor of the subject (“tumor-associated PTP” or “TA-PTP”). Such a TA-PTP has been identified by inventors as a PTP activating CD8⁺ T cells. In another preferred embodiment this PTP is used as a cancer vaccine in combination with a corresponding full-length protein or polypeptide, i.e. with a protein or polypeptide canonically translated by the same mRNA, or with an antigen thereof.

Another object of the invention is a nucleic acid sequence (DNA or mRNA) encoding a PTP as herein defined for use as a vaccine in a subject.

An additional object of the invention relates to a microvesicle, typically an exosome or an equivalent tumor-derived microvesicle, comprising/expressing at least one PTP as herein described.

Exosomes are vesicles of endosomal origin that are secreted in the extracellular milieu following fusion of late endosomal multivesicular bodies with the plasma membrane (Garin et al., 2001; Thery et al., 2002). Cells from various tissue types have been shown to secrete exosomes, such as dendritic cells, B lymphocytes, tumor cells and mast cells, for instance. Exosomes derived from tumor cells are herein identified as tumor-derived microvesicles. Exosomes or tumor-derived microvesicles obtained from melanoma cells are herein identified as “melanosomes”. Exosomes from different origin exhibit discrete sets of proteins and lipid moieties (Thery et al., 1999, Thery et al., 2001). They notably contain proteins involved in antigen presentation and immuno-modulation indicating that exosomes play a role in cell-cell communications leading to the modulation of immune responses. Indeed, exosomes from dendritic cells (DC) pulsed with peptides derived from tumor antigens elicit anti-tumor responses in animal model using the matching tumor (Wolfers et al., 2001, Zitvogel et al., 1998). Methods of producing, purifying or using exosomes for therapeutic purposes or as research tools have been described for instance in WO99/03499, WO00/44389 and WO97/05900, incorporated therein by reference. Recombinant exosomes have been described in the art, which derived from cells transfected with plasmids encoding recombinant proteins. Such recombinant exosomes contain the plasmid-encoded recombinant protein (WO00/28001). Methods of manipulating the protein content of exosomes and of displaying antigens, adjuvant and markers for therapeutic purposes or as research tools have been described in WO03/016522.

Inventors thus herein describe a microvesicle, typically a microvesicle derived from tumor cells, comprising/expressing a PTP as herein defined for use as vaccine, preferably as a cancer vaccine, in a subject. In a particular embodiment, this microvesicle comprises several PTPs, in particular several PTPs of different lengths and optionally of different origin, i.e. derived from distinct (non-spliced) mRNA.

The tumor-derived microvesicles produced by tumor cells may be collected and/or purified according to techniques known in the art, such as by centrifugation, chromatography, etc. Preferred techniques have been described in WO00/44389 and in U.S. Ser. No. 09/780,748, incorporated herein by reference.

Inventors also herein describe a method of preparing functionalized microvesicles/exosomes/melanosomes-containing/expressing a PTP as herein described, the method comprising:
providing a chimeric genetic construct encoding a PTP;
introducing said construct into microvesicles/exosomes/melanosomes-producing cells to generate functionalized microvesicles/exosomes/melanosomes-containing/expressing said PTP, typically presenting said PTP at their surface, and
collecting and/or purifying said functionalized microvesicles/exosomes/melanosomes.

The microvesicles produced by such cells may be collected and/or purified according to techniques known in the art, such as by centrifugation, chromatography, etc. Preferred techniques have been described in WO00/44389 and in U.S. Ser. No. 09/780,748, incorporated herein by reference.

Inventors further herein describe a method of producing a PTP as herein described, the method comprising:
providing a chimeric genetic construct encoding a PTP;
introducing said construct into microvesicles/exosomes/melanosomes-producing cells to generate functionalized microvesicles/exosomes/melanosomes-containing/expressing said PTP, typically presenting said PTP at their surface,
collecting and/or purifying said functionalized microvesicles/exosomes/melanosomes, and
recovering and/or purifying said polypeptide or a fragment thereof from said functionalized microvesicles/exosomes/melanosomes.

Within the context of this invention, the term microvesicles (exosomes or melanosomes) that “comprise/expresse” an antigenic epitope derived from PTP or a PTP designates microvesicles that contain such antigenic epitope derived from PTP or PTP attached to their membrane. The antigenic epitope derived from PTP may be exposed outside of the microvesicle, and the PTP is typically contained within the microvesicle (i.e., attached to the inner side of the membrane or in suspension inside the microvesicle). Typically, the microvesicle allows efficient transport of the PTP(s) to the dendritic cells and allows efficient cross-presentation of the PTP(s) and antigenic epitope(s) derived therefrom at the dendritic cell surface.

This invention further encompasses a vector comprising a chimeric genetic construct as described above, as well as recombinant cells comprising a chimeric genetic construct or a vector as described above.

The vector may be a plasmid, a phage, a virus, an artificial chromosome, etc. Typical examples include plasmids, such as those derived from commercially available plasmids, in particular pUC, pcDNA, pBR, etc. Other preferred vectors are derived from viruses, such as replication defective retroviruses, adenoviruses, AAV, baculoviruses or vaccinia viruses. The choice of the vector may be adjusted by the skilled person depending on the recombinant host cell in which said vector should be used. In this regard, it is preferred to use vectors that can transfect or infect mammalian cells. Indeed, preferred recombinant host cells are mammalian cells. These can be primary cells or established cell lines. Illustrative examples include fibroblasts, muscle cells, hepatocytes, immune cells, etc., as well as their progenitor or precursor cells. Most preferred mammalian cells are exosome-producing mammalian cells. These include, for instance, tumor cells, dendritic cells, B and T lymphocytes or mastocytes.

The microvesicle of the invention can be used alone as a vaccine. In a preferred embodiment, this microvesicle is used in combination with a full length protein or polypeptide expressed by a target cell or tissue (for example tumor) and/or with at least one PTP, typically with several PTPs, derived from the non-spliced mRNA corresponding to said full-length protein or polypeptide.

An additional object of the invention concerns a composition, in particular a vaccine composition, preferably a cancer vaccine, comprising a product as herein described, typically at least one PTP, the PTP full-length corresponding protein or polypeptide, and/or a microvesicle (exosomes or melanosomes) as herein described, and a pharmaceutically acceptable carrier or excipient.

A preferred composition of the invention comprises several PTPs of different lengths. Another preferred composition of the invention comprises PTPs activating CD4⁺ T cells and/or CD8⁺ T cells.

When present, the microvesicle typically includes (contains or expresses) PTP(s), for example PTPs identical to that present as such in the composition optionally together with (at least one) distinct PTP(s).

A preferred vaccine composition comprises at least a first PTP as herein described, a microvesicle and a pharmaceutically acceptable carrier or excipient. Preferably, the microvesicle comprises at least one second PTP consisting in a peptide having 7 to 50 amino acids, said second PTP preferably comprising at least one MHC class I epitope and/or at least one MHC class II epitope, the microvesicle optionally comprising the first PTP.

The microvesicles can be a composition of microvesicles comprising recombinant microvesicles expressing desired PTP(s) and natural microvesicles derived from the subject to be treated, for example microvesicles derived from the tumor of the subject to be treated (tumor-derived microvesicles).

In a preferred embodiment, microvesicles are CD8⁺ T cells activating microvesicles, typically exosomes or tumor-derived microvesicles, such as melanosomes, naturally expressing PTPs activating CD8⁺ T cells of the subject having the tumor.

In another distinct embodiment of the invention, the composition is a vaccine composition comprising a nucleic acid sequence (DNA or mRNA) or genetic construct encoding a PTP as herein defined.

Genetic vaccination can be performed using a variety of viral vectors, such as vaccinia, pox virus, adenovirus, adeno associated virus, etc., non-viral vectors, such as nucleic acid sequence associated with various lipidic or peptidic compositions, or using pure (e.g., naked or in other words free of any transfection facilitating agent) nucleic acid. Vaccination may be performed through various routes of injections, including intra muscular, intra-venous, subcutaneous or intra-dermal. Various vector delivery devices or techniques may be used for genetic vaccination, including gene gun or electroporation. The subject may also be immunized using cell lines transfected in vitro with the vectors. Cell lines selected for release of high number of exosomes would be particularly advantageous.

A preferred cancer vaccine comprises tumor-associated PTP(s) together with exosomes, preferably tumor-derived microvesicles, and/or the PTP full-length corresponding protein or polypeptide, and a pharmaceutically acceptable carrier or excipient.

Another preferred cancer vaccine comprises PTPs and microvesicles both derived from the tumor of the subject to be vaccinated, preferably together with at least one distinct PTP and/or with exosomes expressing the same PTPs and/or at least one distinct PTP, and a pharmaceutically acceptable carrier or excipient. A further preferred cancer vaccine additionally comprises the PTP full-length corresponding protein or polypeptide.

A pharmaceutically acceptable excipient, vehicle or carrier, usable in the context of the present invention, is for example a saline, diluent, isotonic, or buffered solution such as Mannitol 20%, optionally combined with stabilizing agents such as isogenic albumin or any other stabilizing protein, glycerol, etc.

Examples of suitable adjuvants include CpG oligodeoxynucleotides, Apoptosis-Inducing Factor (AIF), Heat Shock Protein (HSP), Toll-like Receptors (TLRs) such as TLR3 agonists (Poly I:C), and cytokines and chemokines such as IL-7, IL-12, IL-15 and Granulocyte Macrophage Colony Stimulating Factor (GM-CSF).

The present invention also relates to the use of a product of the invention as herein described (PTP, microvesicle, nucleic acid) for preparing a composition, in particular a vaccine composition, for preventing or treating a disease, in particular a cancer, in a subject. A typical vaccine composition is for use in a human being.

An object of the invention also relates to a method of producing an immune response in a subject, typically of vaccinating a subject, against a specific target, preferably a tumor antigen or cancer/tumor cell, the method comprising injecting to said subject a PTP according to the invention derived from said target, a microvesicle according to the invention including said PTP, or a vaccine composition according to the invention.

Another object of the invention is a method of preventing or treating a cancer in a subject, the method comprising injecting to said subject a PTP according to the invention, preferably a PTP derived from a protein or polypeptide expressed by the cancerous tumor of the subject, a microvesicle according to the invention including said PTP, or a vaccine composition according to the invention.

As used herein, “treatment” or “treat” refers to therapeutic intervention in an attempt to alter the natural course of the subject being treated, and can be performed either for preventive (prophylactic) or curative purpose. Desirable effects of treatment include, but are not limited to, preventing occurrence or recurrence of disease, alleviation of symptoms, and diminishment of any direct or indirect pathological consequences of the disease, decreasing the rate of disease progression, amelioration or palliation of the disease state, and remission or improved prognosis. In preferred embodiments, compositions and methods of the invention are used to delay development of a cancer or to slow the progression of a cancer, typically of tumor growth.

Typically, the treatment will induce a therapeutic response of the immune system of the subject, typically CD4⁺ and/or CD8⁺ T cells response(s). By inducing a T cell response is meant herein that a T cell response directed towards a certain antigen is elicited. Before said induction, said T cell response was not present, or below detection levels or not functional. By enhancing a T cell response is meant herein that the overall action of T cells directed towards a certain antigen is made higher and/or more efficient compared to the overall action of said T cells before said enhancement. For instance, after said enhancement more T cells directed towards said antigen may be generated. As a result, the action of the additionally generated T cells increases the overall action against said antigen. Alternatively, said enhancement may comprise the increment of the action of T cells directed towards said antigen. Said T cells may for instance react stronger and/or quicker with said antigen. Of course, the result of said enhancement may be generation of additional T cells together with increment of the action of said T cells. Alternatively, said enhancement may comprise generation of additional T cells, or increment of the action of T cells, only.

The treatment, typically vaccine, is intended for a subject. The term “subject” or “individual” refers to an animal, typically a mammal. Examples of mammals include humans and non-human animals such as, without limitation, domesticated animals (e.g., cows, sheep, cats, dogs, and horses), non-human primates (such as monkeys), rabbits, and rodents (e.g., mice and rats). The treatment is preferably intended for a human being in need thereof, whatever its age or sex. Are in particular considered as such, the subjects suffering of a cancer, or those considered “at risk of developing” such a cancer, in which this has to be prevented. The patient typically has a tumor. Unless otherwise specified in the present disclosure, the tumor is a cancerous or malignant tumor.

The cancer or tumor may be any kind of cancer or neoplasia. The tumor is typically a solid tumor, in particular of epithelial, neuroectodermal or mesenchymal origin. It can be selected from a melanoma, a sarcoma, a carcinoma, a lymphoma, and a paediatric tumour (glioma), for example from a melanoma or sarcoma. The invention is applicable, in the context of therapy, to primary tumors, or secondary invasions, loco-regional or distant metastases, and in the context of prophylaxis, in order to avoid secondary malignant central nervous system involvement such as the observed invasions (metastasis) from melanoma, lung cancer, kidney cancer, breast cancer, and colon cancer.

In the vaccine composition of the invention, PTPs are present in an amount sufficient to elicit a therapeutic response of the immune system of a given subject against a desired target (pathogen, target cell), for example a CD8⁺ T cells response, typically a CD4⁺ T cells response, preferably CD4⁺ and CD8⁺ T cells therapeutic responses, and prevent or treat, typically control, a disease, preferably a cancer.

When the subject is a mammal, preferably a human being, the vaccine composition typically comprises from 0.1 to 10 mg per kg of body weight of PTPs, optionally together with 0.1 to 5 mg per kg of body weight of microvesicles.

The herein described products capable of inducing a therapeutic immune response may be administered in vivo to any mammalian subject in need thereof, in particular human subjects. Administration can be performed by various routes, such as by systemic injection, e.g., intravenous, intra-muscular, intra-peritoneal, intra-tumoral, sub-cutaneous, etc.

The detection of a therapeutic immune response can be easily determined by the skilled person thanks to technologies such as ELISA, ELISPOT, delayed type hypersensitivity response, intracellular cytokine staining, and/or extracellular cytokine staining.

The invention will be further illustrated by the following figures and examples. However, these examples and figures should not be interpreted in any way as limiting the scope of the present invention.

### FIGURES

FIG. 1: Role of the Pioneer Translation Products (PTPs) in tumor rejection.

A) Mice were injected subcutaneously with either MCA205 WT tumor cells or MCA205 transfected by the plasmid coding for glob-intron-SL8, the plasmid coding for glob-exon-SL8 or Ovalbumin. Half of the mice from each group received intravenous OT1 cells at Day 6 or Day 4. Tumor size was assessed through time until day 20. Data are given as mean±SEM. * p<0.05 (unpaired student t test).

B) Mice were injected subcutaneously with B16F10 WT tumor cells or B16F10 transfected by the plasmid coding for glob-intron-SL8, the plasmid coding for glob-exon-SL8 or Ovalbumin. At Day 3, half of the mice from each group received intravenous OT1 cells. Tumor size was assessed through time until day 19. Data are given as mean±SEM. * p<0.05 (unpaired student t test).

C) Mice were injected intraveinously with 2·10⁶ OT1 cells marked with CFSE. After 3 h, 5·10⁶ Hek cells WT or transfected by the plasmid glob-intron-SL8 or glob-exon-SL8 or Ova were injected intraperitoneally. After 3 days, cells from the lymph nodes and the spleen were collected and the CFSE expression in CD8 cells was analyzed. The dot plots are representative of the results obtained in the different mice.

FIG. 2: All PTPs: source of peptides for cancer-vaccines.

Groups of 6 mice were vaccinated with 125 μg (PTPs×1), 64 μg (PTPs×1/2), 32 μg (PTPs×1/4) of PTPs or with 8 μg (SIINFEKL 1/25) of SIINFEKL epitope (positive control for the MCA-205-Ova and negative control for the MCA-205 WT cells) emulsified in CpG+poly I:C (negative control). 15 days later, mice were challenged subcutaneously with 50.10³ MCA-205 living cells expressing Ovalbumin in the right flank (A) and with 50.10³ MCA-205 WT living cells in the left flank (B). The tumor growth was measured every 7 days for each tumor cell lines. Each line represents the tumor size in area (mm²) of the 6 mice in each group.

FIG. 3: specific PTPs from sarcoma cell lines: source of peptides for cancer-vaccines.

Groups of 6 mice were vaccinated with 125 μg (PTPs-his ×1), 64 μg (PTPs-his ×1/2), 32 μg (PTPs-his ×1/4) of PTPs or with 8 μg (SIINFEKL 1/25) of SIINFEKL epitope (positive control) emulsified in CpG+poly I:C (negative control). 15 days later the mice were challenged subcutaneously with 50·10³ MCA-205 living cells expressing Ovalbumin in the right flank (A) and with 50·10³ MCA-205 WT living cells in the left flank (B). The tumor growth was measured every 7 days for each tumor cell lines. Each line represents the tumor size in area (mm²) of the 6 mice in each group.

FIG. 4: PTPs plus exosomes: a new cancer-vaccine.

A) Analysis of the expression of CD9 and CD81 in exosomes purified from MCA205-glob-intron-SL8 cells by FACS. In pale grey the unstained exosomes, in dark grey the WT exosomes and in black the glob-intron-SL8-exosomes.

B) Left Panel: BMDCs (bone marrow dendritic cells) were pulsed by exosomes purified from MCA205 WT or MCA205-glob-intron-SL8. They were collected and cultured with OT1 cells. An ELISA to detect IL-2m was performed. Data are given as mean±SEM. Right panel: Exosomes were added to OT1 cells in absence of BMDCs. The quantity of mIL-2 produced in the supernatant after at least 18 h was evaluated by ELISA. The data are expressed as mean±SEM.

C) FACS analysis of the expression of SIINFEKL using the 25D1 antibody on the MCA205 cells and exosomes. Left panel, in dashed line the unstained MCA205 cells, in pale grey the WT MCA205 and in white MCA205 cells expressing the Glob-intron-SL8 construct. Right panel, in pale grey the unstained exosomes, in dark grey exosomes purified from MCA205 cells and in black the exosomes purified from MCA205 cells expressing the Glob-intron-SL8 construct.

D) Groups of 6 mice were vaccinated with 64 μg (PTPs-his ×1/2), 32 μg (PTPs-his ×1/4) of tumor-derived PTPs or with 64 μg (PTPs-his ×1/2), 32 μg (PTPs-his ×1/4) of tumor-derived PTPs plus 15 μg of tumor-derived exosomes containing PTPs, or as positive control 8 μg (SIIN 1/25) of SIINFEKL epitope emulsified in CpG+poly I:C. 15 days later, the mice were challenged subcutaneously with 50·10³ MCA-205 living cells expressing Ovalbumin in the right flank. The tumor growth was measured for each tumor cell lines every 7 days. Each line represents the tumor size in area (mm²) of the 6 mice in each group.

FIG. 5: Addition of CD4 epitope to improve the PTPs cancer-vaccine.

Groups of 6 mice were vaccinated with 64 μg (PTPs-his ×1/2) of tumor-derived PTPs or with 64 μg (PTPs-his ×1/2) of tumor-derived PTPs plus 1 mg of purified Ovalbumin, or as positive control 8 μg (SIIN 1/25) of SIINFEKL epitope emulsified in CpG+poly I:C. 15 days later, the mice were challenged subcutaneously with 50·10³ MCA-205 living cells expressing Ovalbumin in the right flank and with 50·10³ MCA-205 WT living cells in the left flank. The tumor growth was measured every 7 days for each tumor cell lines. Each line represents the tumor size in area (mm²) of the 6 mice in each group.

FIG. 6: Figure illustrating the different positions of the SL8 antigenic epitope in the Ovalbumin cDNA and in the introns sequence of the β-Globin gene.

FIG. 7: specific PTPs from melanoma cell lines: source of peptides for cancer-vaccines.

Groups of 6 mice were vaccinated with 32 μg or 16 μg of PTPs-His or with 8 μg of SIINFEK1 epitope (positive control) emulsified in CpG+Poly I:C (negative control). Fifteen days later the mice were challenged subcutaneously with 30·10³ B16F10 living cells expressing Ovalbumine in the right flank along with matrigel (A) and with 30·10³ B16F10 WT living cells in the left flank (B). The tumor growth was measured every 3-4 days for each tumor cell lines. Each line represents the average tumor size in area (mm²) of the 6 mice in each group.

FIG. 8: PTPs plus exosomes from melanoma cell lines.

Groups of 6 mice were vaccinated with 16 μg of PTPs-His, with 15 μg of exosomes derived from B16F10 cells or with PTPs-His 16 μg along with 15 μg exosomes emulsified in CpG+Poly I:C (negative control). Fifteen days later the mice were challenged subcutaneously with 30·10³ B16F10 living cells expressing Ovalbumine in the right flank along with matrigel (A) and with 30·10³ B16F10 WT living cells in the left flank (B). The tumor growth was measured every 3-4 days for each tumor cell lines. Each line represents the average tumor size in area (mm²) of the 6 mice in each group.

FIG. 9: PTPs plus melanosomes from melanoma cell lines.

A) BMDCs were pulsed by melanosomes purified from B16F10-glob-intron-SL8 cells. The BMDCs were then co-culture with the SL8-specific CD8+ T-cell hybridoma (B3Z) for 16 h and T-cell activation was estimated by measuring β-galactosidase. B and C) Groups of 6 mice were vaccinated with 32 μg of PTPs-His or with 30 μg of melanosomes derived from B16F10 cells emulsified in CpG+Poly I:C (negative control). Fifteen days later the mice were challenged subcutaneously with 30·10³ B16F10 living cells expressing Ovalbumine in the right flank along with matrigel (B) and with 30·10³ B16F10 WT living cells in the left flank (C). The tumor growth was measured every 3-4 days for each tumor cell lines. Each line represents the average tumor size in area (mm²) of the 6 mice in each group.

Throughout this application, various references describe the state of the art to which this invention pertains. The disclosures of these references are hereby incorporated by reference into the present disclosure.

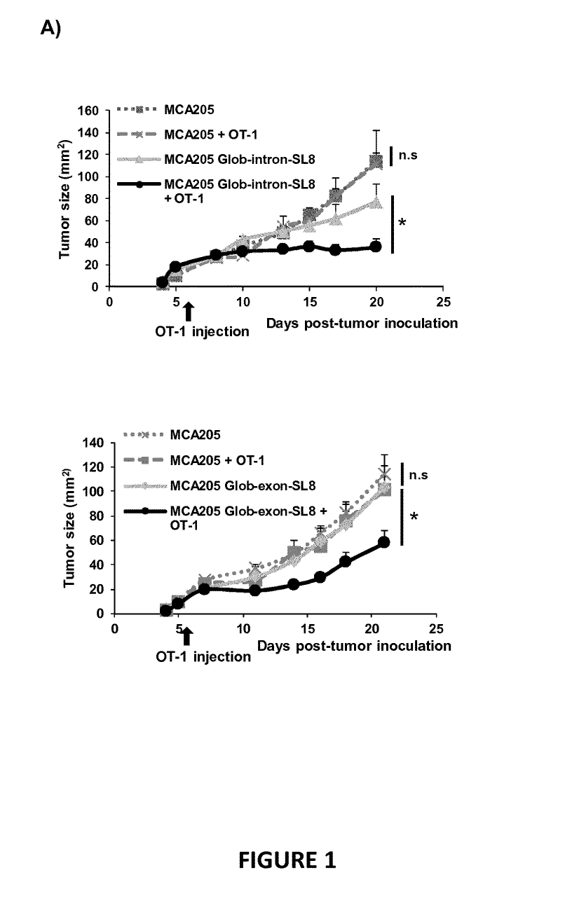

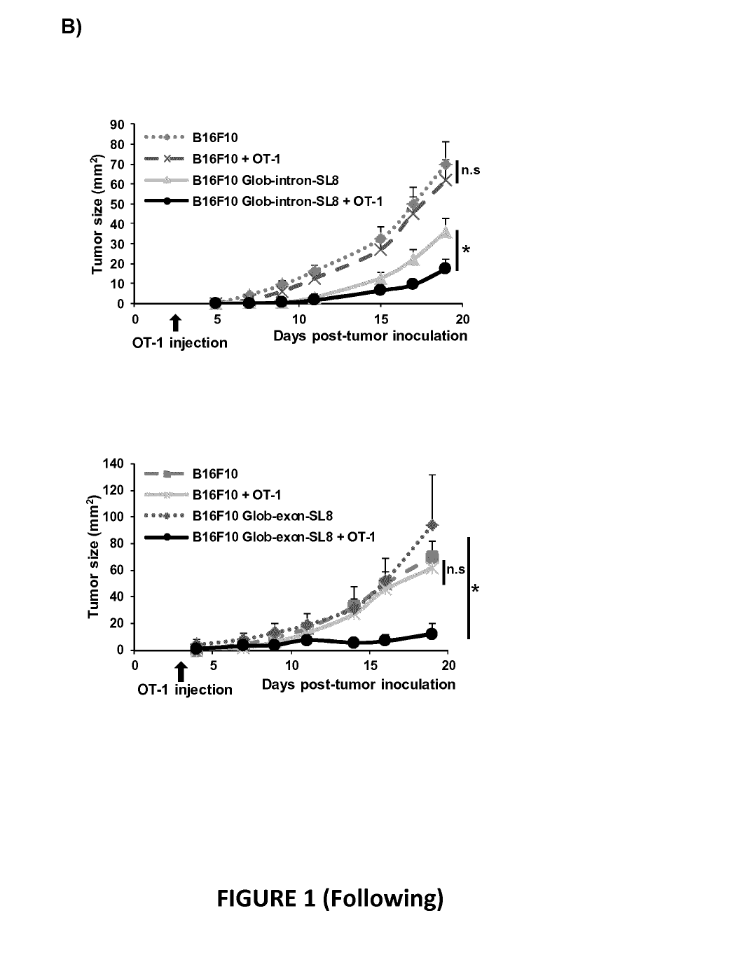

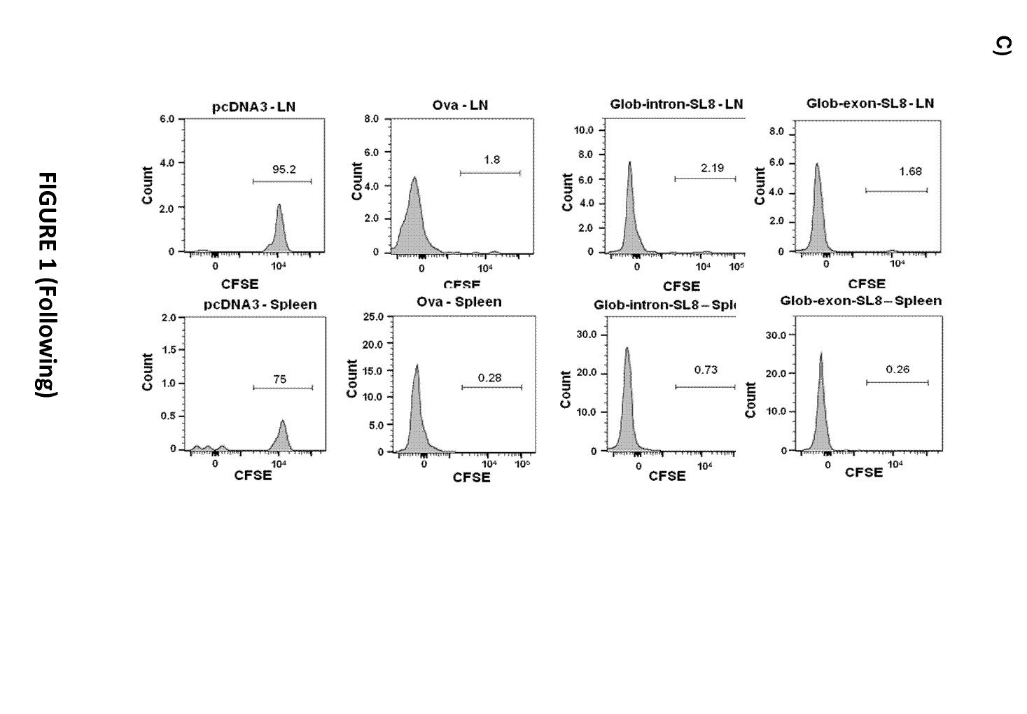

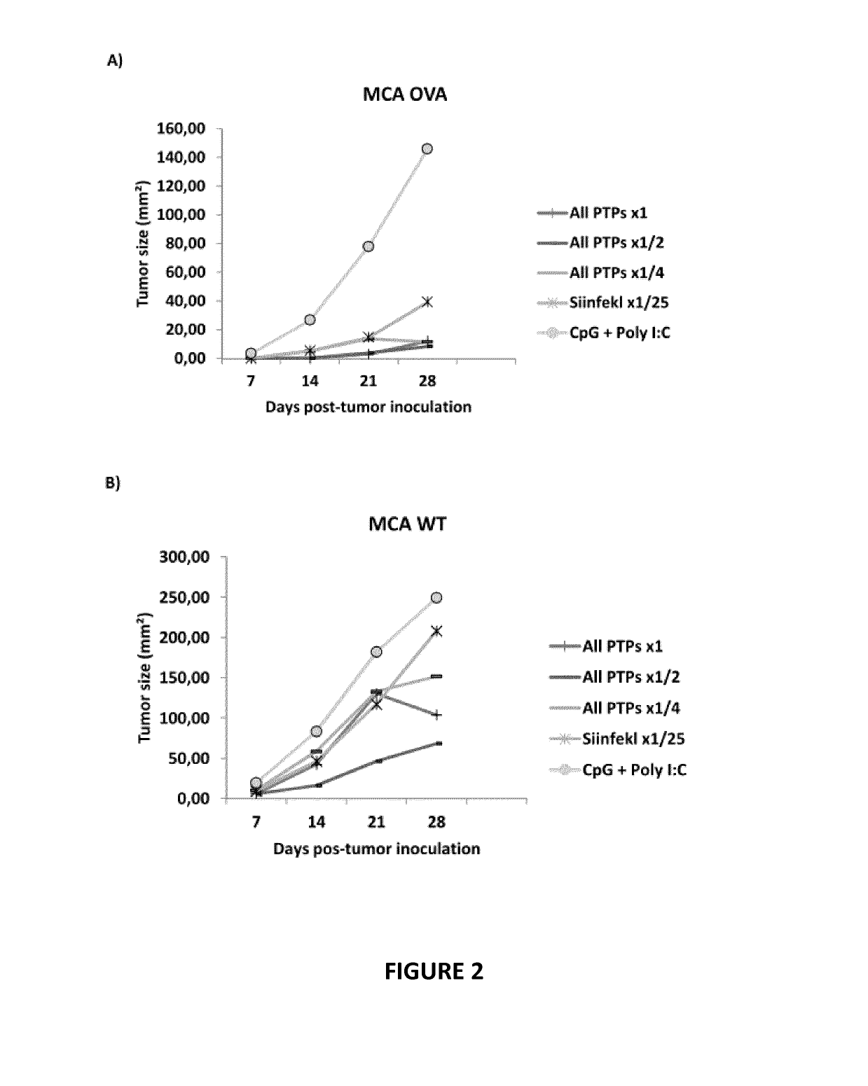

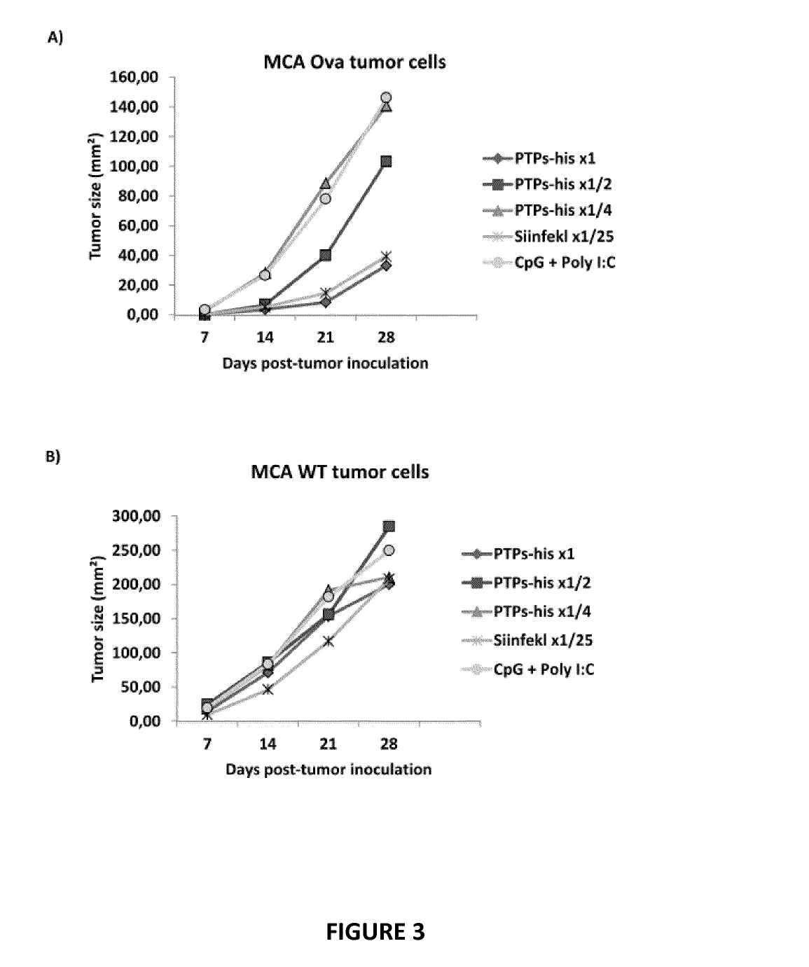

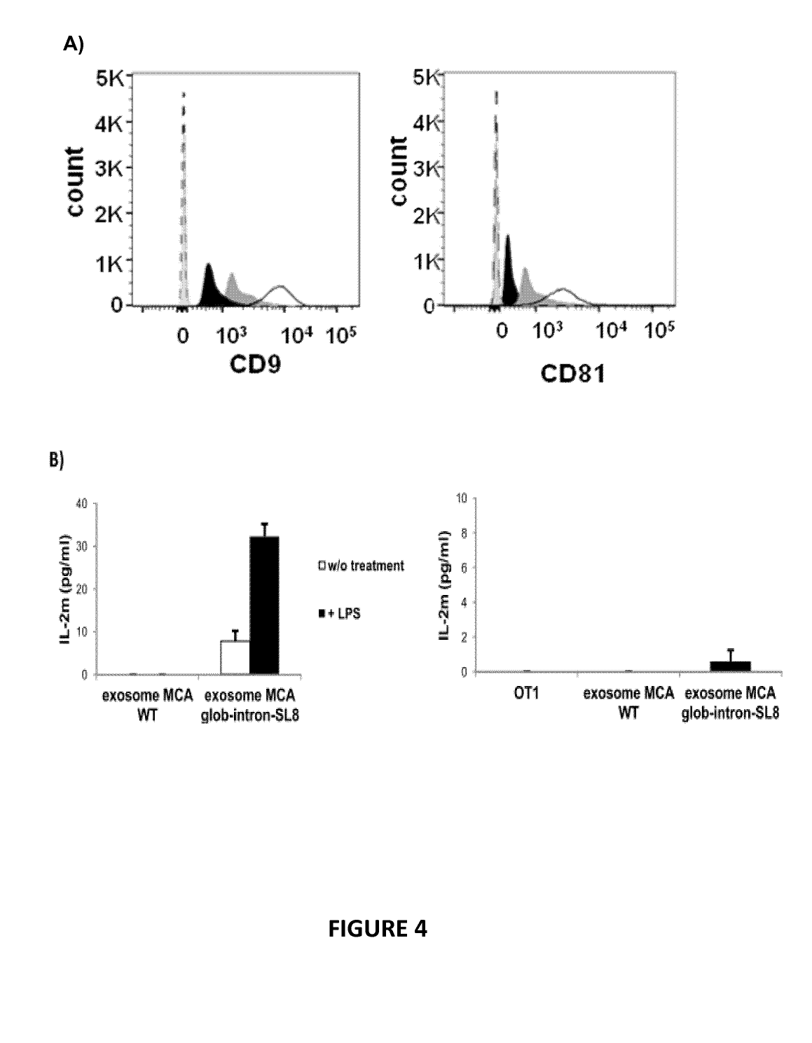

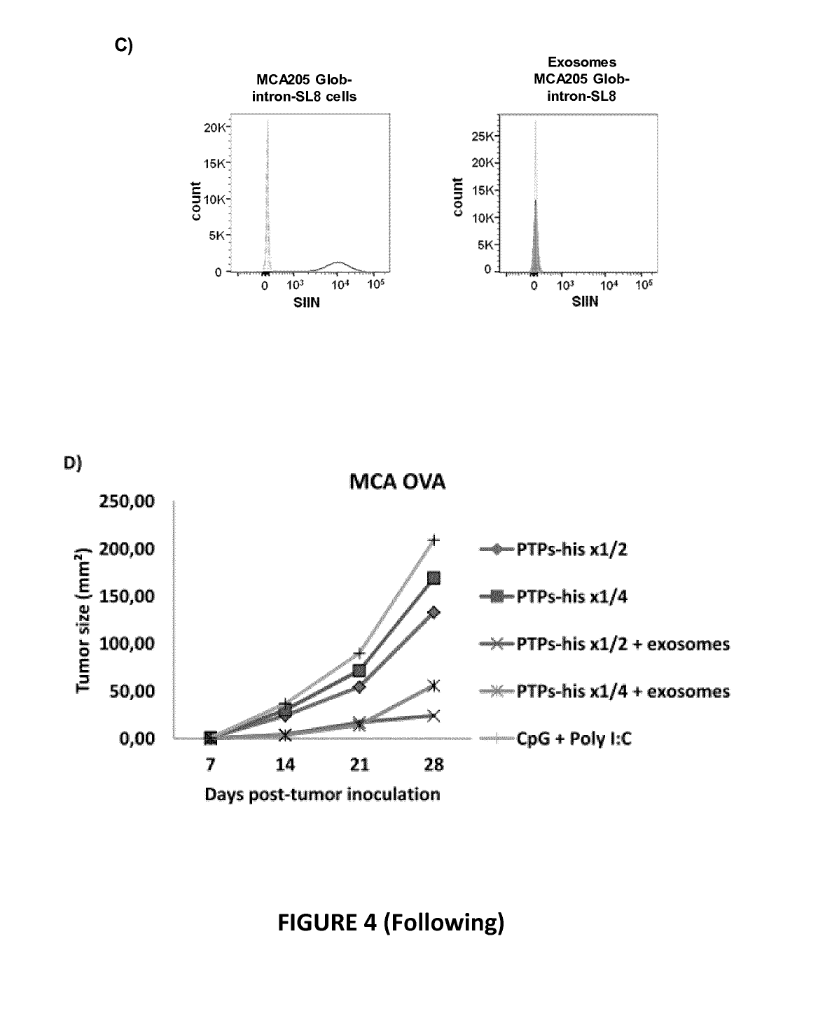

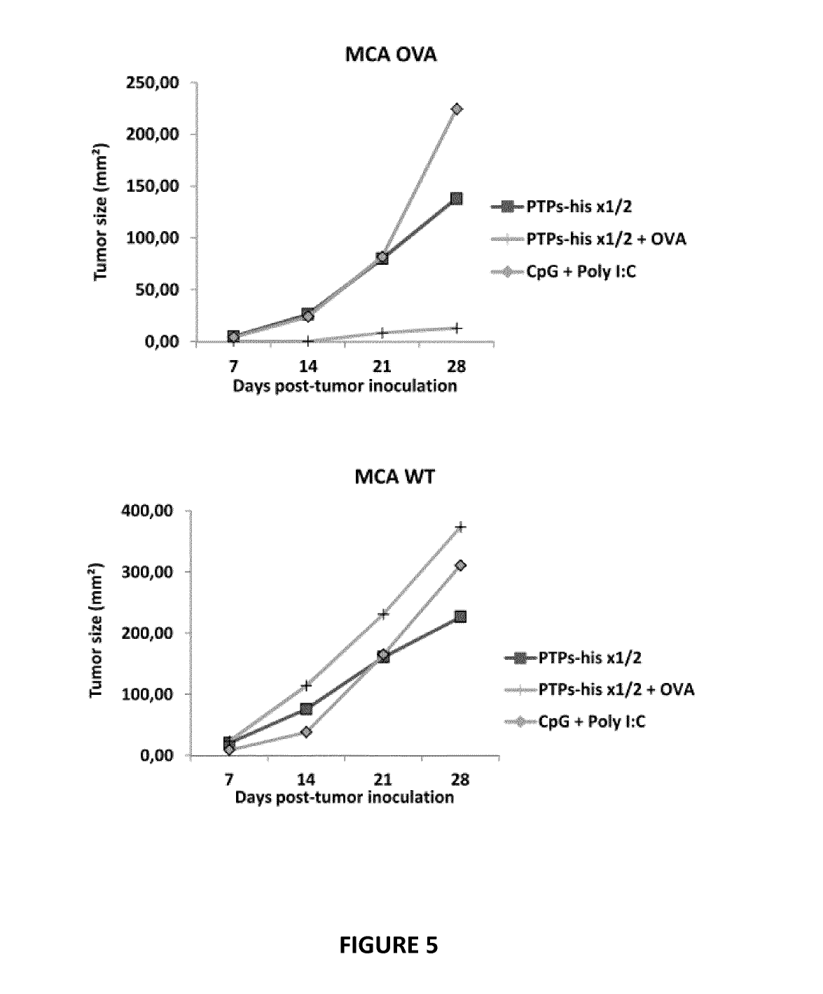

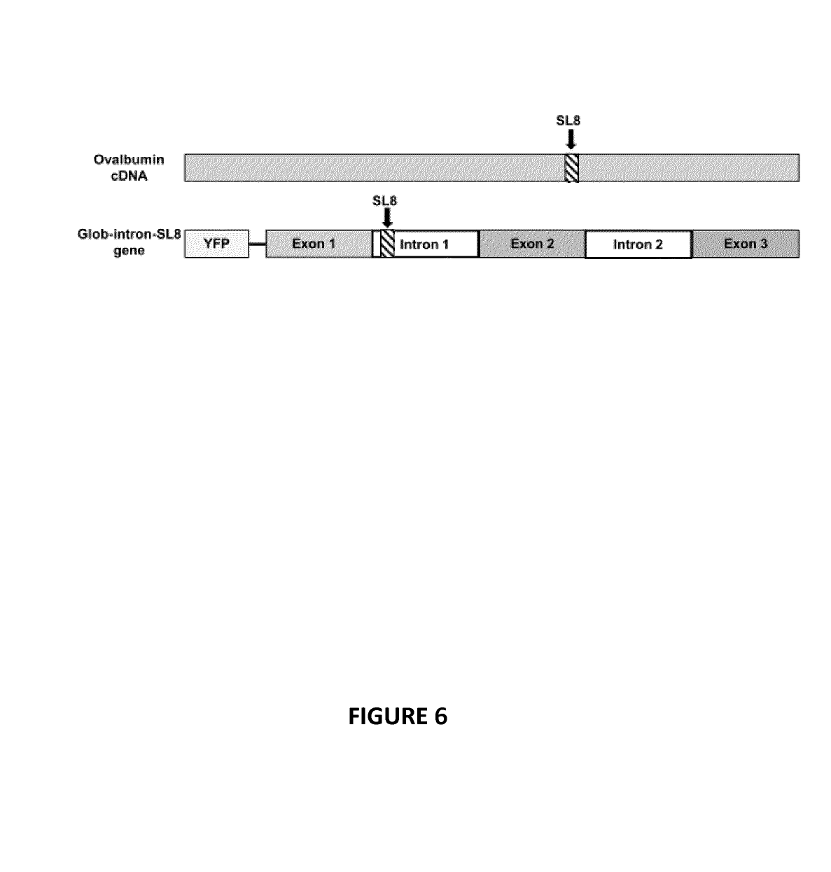

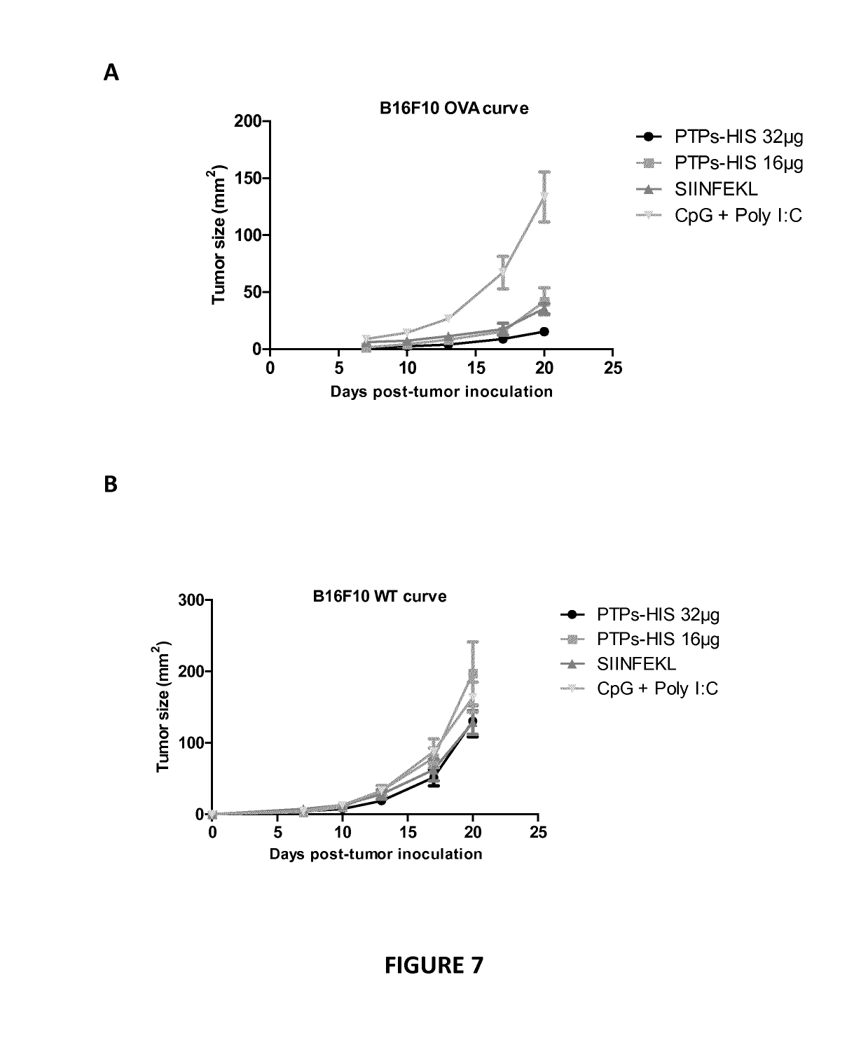

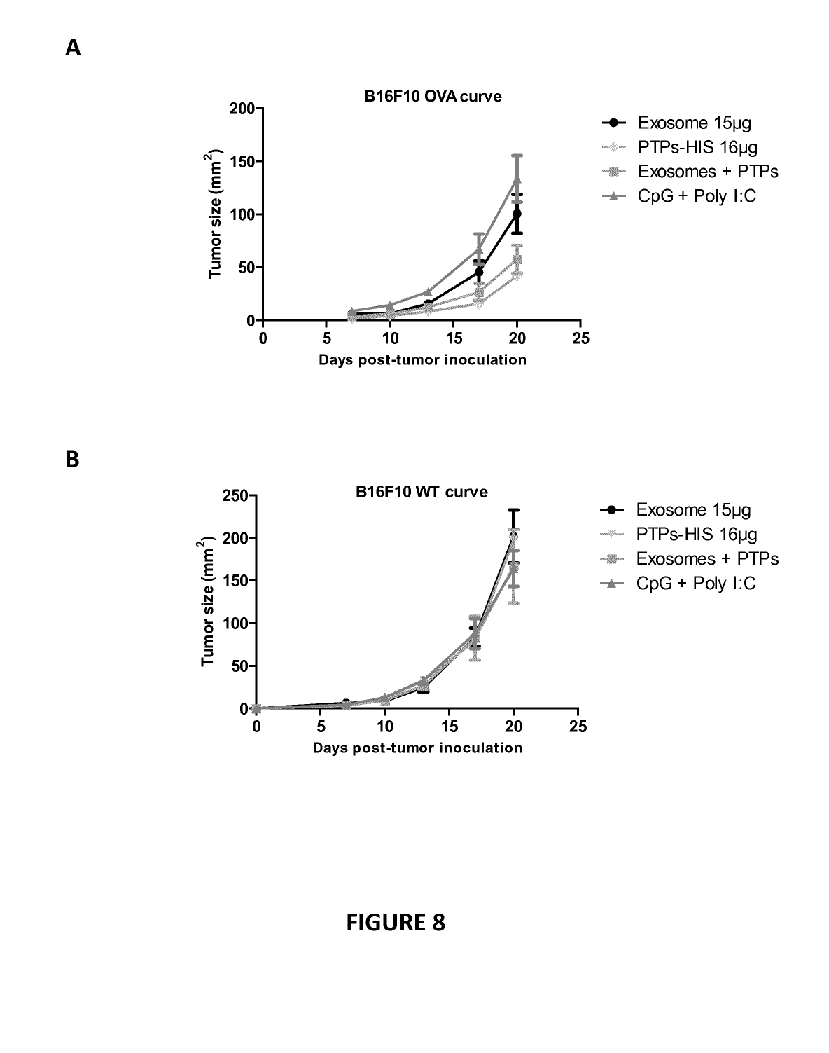

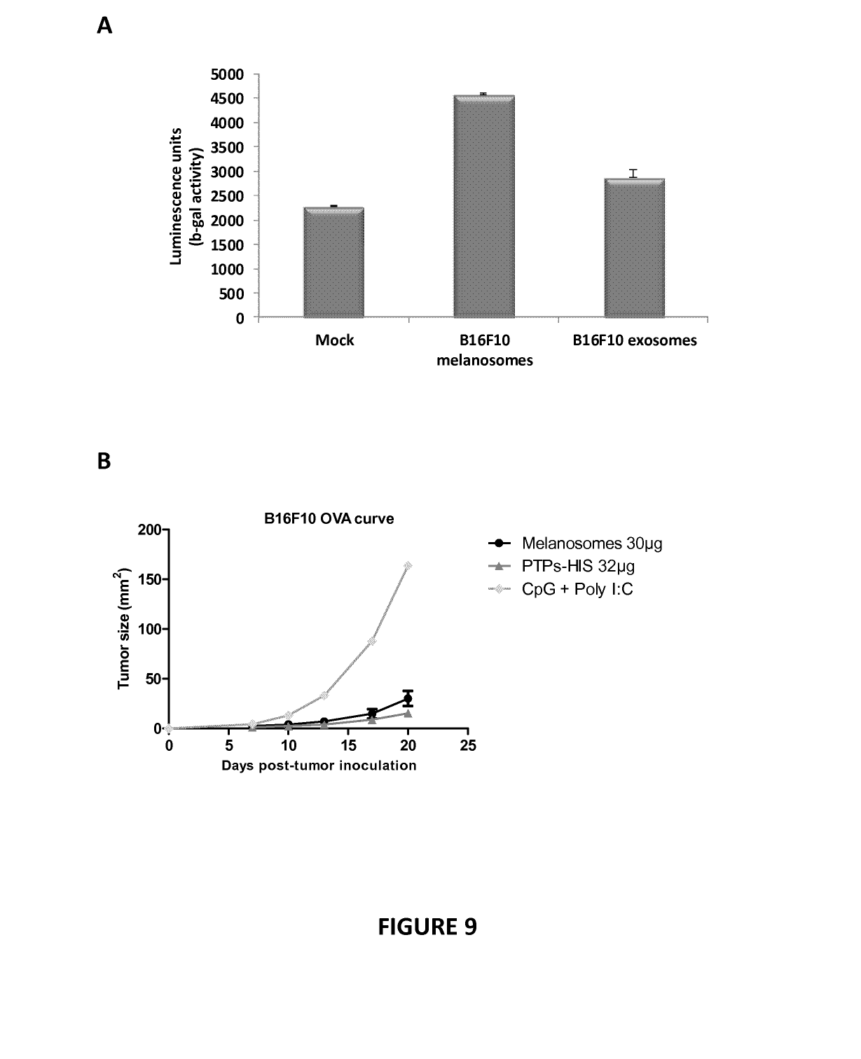

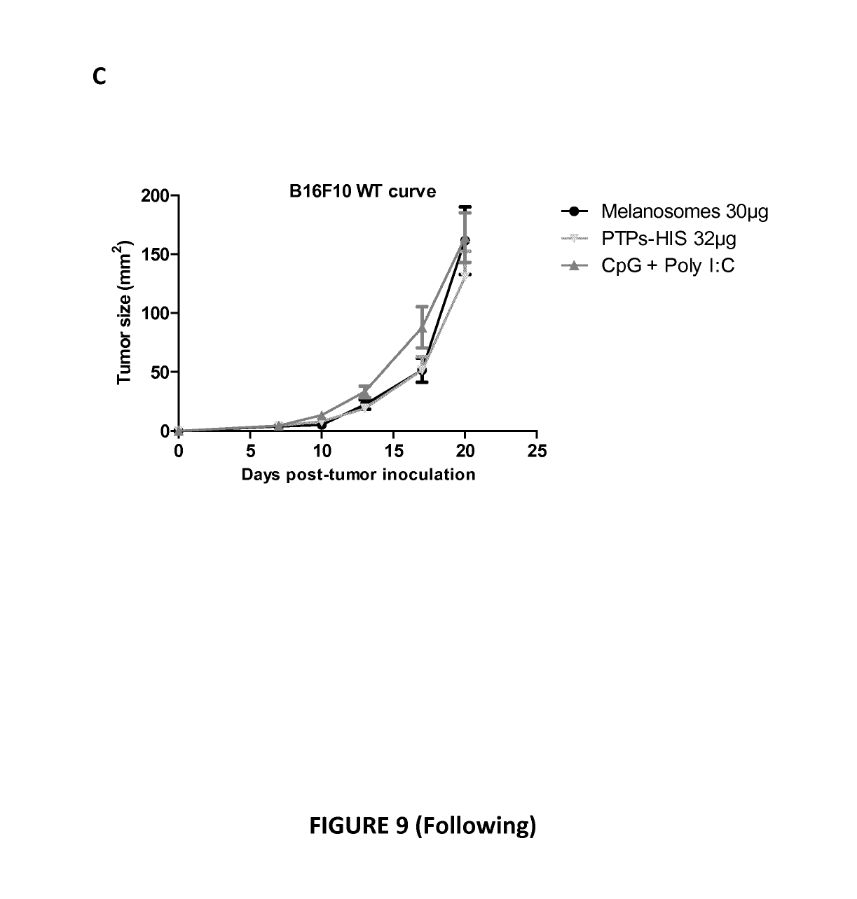

Other characteristics and advantages of the invention are given in the following experimental section (with reference to FIGS. 1 to 6), which should be regarded as illustrative and not limiting the scope of the present application.

### EXPERIMENTAL PART

### Example 1—Pioneer Translation Products (PTPs) in Combination with Exosomes: A New Cancer Vaccine

### Materials and Methods

### Cell Culture

MCA 205 mouse sarcoma cell line were cultured at 37° C. under 5% CO₂ in RPMI 1640 medium (Life Technologies) in the presence of 1% glutamine, 1% pyruvate, 1% non-essential amino-acids and 10% FBS (Life Technologies) under standard conditions. B16F10 (syngeneic from C57BL/6J mice) were cultured at 37° C. under 5% CO₂ in DMEM containing 10% FCS, 2 mM L-glutamine and 100 IU/ml penicillin/streptomycin.

MCA 205 and B16F10 cells were transfected with YFP-globine-intron-SL8-his plasmid using JetPrime according to the manufacturer's protocol (Ozyme) for the purification of PTPs. For the tumor rejection experiment, stable MCA 205-Ova and stable B16F10-Ova cells were prepared. Stable MCA 205-Ova are cultured in RPMI 1640 under standard conditions. Stable B16F10-Ova cells stably expressing the Ovalbumin protein are cultured in DMEM under standard conditions.

### Animal Studies.

C57B1/6J mice were obtained from Harlan. OT1 C57B1/6J mice were generously provided by the CERFE (C. Daviaud) and bred at Gustave Roussy animal facility. 7 weeks C57BL/6J mice were inoculated with 0.1×10⁶ MCA205 or B16F10 tumor cells subcutaneously in the right flank. For MCA205, when the tumors reached a size around 20 mm², the mice were injected with 0.1×10⁶ OT1 cells intravenously. In the B16F10 model, 0.2×10⁶ OT1 cells were inoculated intravenously three days after the tumor inoculation. All animal experiments were carried out in compliance with French and European laws and regulations.

### PTPs-his Purification

Transfected MCA 205 or B16F10 tumor cells were sonicated in 10 mL of 6M guanidium-HCl, 0.01M Tris/HCl, pH 8.0, 5 mM imidazole and 10 mM β-mercaptoethanol. Then, the lysate was incubated and rotated with Ni²⁺-NTA-agarose beads (Qiagen) for 4 h at RT. The beads were washed successively for 5 min at RT with 8 mL of each of the following buffers: 6M guanidium-HCl, 0.01M Tris/HCl, pH 8.0 and 10 mM β-mercaptoethanol; 6M urea, 0.01M Tris/HCl, pH 8.0 and 10 mM β-mercaptoethanol; 6M urea, 0.01M Tris/HCl, pH 6.8, 10 mM β-mercaptoethanol and 0.2% Triton X-100; 6M urea, 0.01M Tris/HCl, pH 6.8 and 10 mM β-mercaptoethanol; 6M urea, 0.01M Tris/HCl, pH 6.8, 10 mM β-mercaptoethanol and 0.1% Triton X-100. PTPs were then eluted by incubating the beads for 20 min at RT in 400 mM imidazole, 0.15M Tris/HCl, pH 6.8, 30% glycerol, 0.72M β-mercaptoethanol and 5% SDS. The eluate was dialyzed in PBS using a dialysis tubing MWCO 0.5 kD (VWR) overnight at RT. Finally, the eluate was quantified by a Bradford assay (ThermoFisher).

### All PTPs Purification

MCA 205 tumor cells were lysed then sonicated in 10 mL of 6 M guanidium-HCl, 0.01 M Tris/HCl, pH 8.0, 5 mM imidazole and 10 mM β-mercaptoethanol. The lysates were purified and the polypeptides were concentrated using a 3 kDa centrifugal filter (Merck Millipore). This column was centrifugated for 90 min. at 3000 g which allow us to purify small polypeptides, the definition of PTPs. The lower part was dialyzed in PBS using a dialysis tubing MWCO 0.5 kDa (VWR) overnight at RT. Finally, the eluate was quantified by a Bradford assay (ThermoFisher).

Peptides Extraction from Solid Tumor

Solid tumor disintegration was performed on ice by crushing material with a 0.22 μm cell strainer. Solubilization was done by the addition of 1×SDS buffer (0.125 M Tris-HCl (pH 6.8), 2% sodium dodecyl sulfate, 10% glycerol, 5% 2-mercaptoethanol) ten times the weight of the tissue. The disintegrated tissue was incubated at 70° C. and was shacked at 1 400 rpm for 10 min. Then, it was centrifuged at 13 200 g for 5 min at RT in order to sediment and eliminate solid tissue. The D-Tube™ Dialyzers (MerckMillipore) was used to purify and concentrate peptides with a molecular weight cut-offs of 5 kDa. A centrifugation was performed at 3 000 g for 1 h30. Finally, the peptide concentration was measured using BCA Protein Assay kit (Pierce).

### Vaccination

Vaccines for the MCA205 cells were prepared according to the following groups: PTPs-his ×1 (128 μg), PTPs-his ×1/2 (64 μg), PTPs-his ×1/4 (32 μg) −/+exosomes, exosomes (purified from MCA transfected cells (15 μg)), PTPs-his ×1/2 (64 μg) −/+(1 mg/50 μL/mouse) Ovalbumin protein (Calbiochem), all PTPs ×1 (128 μg), all PTPs ×1/2 (64 μg), all PTPs ×1/4 (32 μg), CpG (20 μg) (Invivogen) and Poly(I:C) (50 μg) (Invivogen), PBS (up to 300 μL). Vaccines were prepared 2 h before injection and kept on ice. Prior to vaccination, C57BL/6 mice were anesthetized with 3% isoflurane. The vaccines were injected subcutaneously in the legs (150 μL/leg) and in footpad (50 μL/foot). Two weeks later, subcutaneous injections of 50*10³ MCA 205 tumor cells (right flank) and MCA 205 OVA tumor cells (left flank) were given. Once a week, tumors size was measured until they reached 300 mm².

Vaccines for the B16F10 cells were prepared according to the following groups: 32 μg or 16 μg of PTPs-His, exosomes (purified from B16F10 transfected cells, 15 ug), melanosomes (purified from B16F10 cells, 30 μg) or with 8 μg of SIINFEKL epitope (positive control), CpG (20 μg) (Invivogen) and Poly(I:C) (50 μg) (Invivogen). Vaccines were prepared 2 h before injection and kept on ice. Prior to vaccination, C57BL/6 mice were anesthetized with 3% isoflurane. The vaccines were injected subcutaneously in the legs (150 μL/leg) and in footpad (50 μL/foot). Two weeks later, subcutaneous injections of 30×10³ B16F10 tumor cells (right flank) and B16F10 OVA tumor cells (left flank) were given. Once a week, tumors size was measured until they reached 300 mm².

### Results

### Role of the Pioneer Translation Products (PTPs) in Tumor Rejection.

In the last decade, PTPs and DRiPs have been proposed to be the major source of peptides for the endogenous MHC class I pathway. To precisely define the role of PTPs in mediating a specific CD8+ T cells anti-tumor immune response, inventors inoculated individual C57BL/6 mice with two different tumor models: the MCA sarcoma model and the B16F10 melanoma model stably expressing their different constructs (see FIG. 6). For the MCA model, 10⁵ tumor cells were subcutaneously injected in C57BL/6 mice. Then tumors were allowed to grow to approximately 20 mm². At this point, 10⁵ naïve Ova-specific TCR-transgenic CD8+ OT-1 T cells were adoptively transferred to the mice. Then tumor growth was monitored and recorded every two days. After 14 days, inventors observed that adoptive transfer of OT-1 T cells prevent the development of MCA tumors stably expressing independently the SIINFEKL/SL8 epitope in the Glob-intron or in the Glob-exon setting (FIG. 1A, down and up panels). And, as expected, adoptive transfer of OT-I T cells do not prevent the growth of SL8-negative MCA tumors (FIG. 1A, down and up panels) confirming the specific recognition of the CD8+ T cells for the antigen that expressed the tumor cell line and confirming the specific role of the PTPs in inducing an anti-tumor response. For the B16F10 model, 10⁵ tumor cells were subcutaneously injected in C57BL6 mice. Then, 3 days later, 10⁵ naïve Ova-specific TCR-transgenic OT-1 cells were adoptively transferred to the mice. Similarly to the MCA model, adoptive transfer of OT-I T cells prevents on one hand the development of B16F10 tumors stably expressing the SIINFEKL/SL8 epitope in the intron or exon sequences (FIG. 1B, up and down panels), and on another hand do not prevents the growth of SL8-negative B16F10 tumors (FIG. 1B, up and down panels) supporting again the idea that PTPs are inducing a specific anti-tumor response.

Moreover, to finally conclude that PTPs can contribute to cross priming in an in vivo model, HEK-293 cells were transfected with the different constructs and injected subcutaneously into CD45.1 congenic C57Bl/6 mice that received, 3 h earlier, naïve OT-I CD8+ T cells stained with CFSE. If PTPs, expressed from exon and/or intron sequences contribute to cross priming then they expected to see a diminution over time of the CFSE fluorescence, demonstrating a proliferation of the CD8+ OT-I T cells. As seen in FIG. 1C, after 3 days of inoculation, the PTPs induced a CD8+ OT-I T cell division as compared to the negative control where HEK-293 cells were only transfected with an empty vector and where CD8+ OT-1 T cells did not proliferated over the same time of inoculation. Since HEK-293 cells are of human origin, they cannot directly present antigen that come from the PTPs directly to the murine CD8+OT-1 T cells. Therefore, the proliferation of the CD8+ T cells can only occur via the cross priming of the PTPs, supporting the tumor rejection results.

These results demonstrate that PTPs can induce a specific immune response in vivo by promoting a specific antigen tumor rejection. Furthermore, these results show that PTPs, in addition to being used as a major source of antigenic peptides for the endogenous pathway, might be also a source of exogenous peptides for the MHC class I exogenous pathway.

### Tumor Polypeptides: Source of Peptides for Cancer-Vaccines

In parallel and to confirm the specific role of polypeptides carrying MHC class I epitopes as being a major source for a cancer vaccine, inventors have purified PTPs from WT tumor cell lines. For that purpose, the MCA205 WT tumor cell lines were lysed and all polypeptides of 5 kDa or smaller than 5 kDa, the definition of a PTP, were purified and used as vaccine in mice, as described previously with PTPs coming from inventors' constructs of interest. Different groups of 6 mice were vaccinated with different concentration of PTPs, or with the adjuvant itself (negative control). After 2 weeks, 50·10⁴ MCA205 tumor cell line, expressing or not the Ovalbumin construct were subcutaneously injected in the right flank (MCA-205 Glob-intron-SL8) and left flank (MCA-205 WT) of mice. Inventors' data indicate that polypeptides of 5 kDa or smaller than 5 kDa purified from the nuclear compartment of a tumor cell lines can induce a defect of the same tumor independently of whether or not the tumor expresses inventors' specific model epitope (FIGS. 2A and 2B).

In parallel, polypeptides from solid tumor that have grown in mice for few weeks were purified. The solid tumors were disintegrated and then the polypeptides containing PTPs were purified with a cut-off of 5 kDa. The purified polypeptides were used as a vaccine in mice challenged two weeks after with the same tumor cell lines from which these polypeptides have been purified. Inventors' data indicate that polypeptides purified from solid tumors can induce a defect of the same tumor independently of whether or not the tumor expresses inventors' specific model epitope.

These experiments shed light the specific effect as a vaccine composed of tumor-derived polypeptides of different lengths, on the growth of the tumor, and, support the idea that PTPs can be used as vaccine to elicit a specific anti-tumor-T-cell response.

### PTPs: Source of Peptides for Cancer-Vaccines

In this study, before being used as a vaccine, PTPs purified from sarcoma MCA205 and melanoma B16F10 cell lines were analyzed by mass spectrometry to look more closely at the nature of the different polypeptides that compose the vaccine. As shown in Table 1, the vaccine consists of different polypeptides of different length.

TABLE 1
Mass spectrometry analysis of peptides derived
from exosomes produced by MCA205 cells. The
peptide corresponding to the SIINFEKL peptide
derived from an intron sequence is highlighted.

| | Peptide | Peptide |
|---|---|---|
| Peptide sequence | length, a.a. | origin |
| VNVDEVGGEALGR | 13 | YFP-globin |
| (SEQ01) | | |
| SAMPEGYVQER | 11 | YFP-globin |
| (SEQ02) | | |
| FEGDTLVNR | 9 | YFP-globin |
| (SEQ03) | | |
| FSVSGEGEGDATYGK | 15 | YFP-globin |
| (SEQ04) | | |
| SIINFEK | 7 | Chicken |
| (SEQ05) | | Ovalbumin |
| LEYNYNSHNVYIMADK | 16 | YFP-globin |
| (SEQ06) | | |
| GEELFTGVVPILVELDGDVNGHK | 23 | YFP-globin |
| (SEQ07) | | |

The SL8 epitope is the epitope that will be recognized at the cell surface by the naïve Ova-specific TCR-transgenic CD8+ OT-1 T cells and will have for consequence to induce a proliferation of specific CD8+ T cells and a tumor rejection.

In the previous part of the study, inventors were looking at tumor rejections of tumor cell lines that were expressing their PTPs with the help of specific CD8+ T cells, whereas in this part of the study they aimed to demonstrate that mice vaccinated with their tumor-derived PTPs in a prophylactic manner show a defect in tumor growth when compared to mice that have not been vaccinated, supporting the hypothesis that PTPs can induce as vaccine a tumor growth defect and a specific CD8+ T cells immune anti-tumor response.

For that purpose, different groups of 6 mice were vaccinated with different concentration of PTPs, or with the adjuvant itself (negative control), or with the SL8 epitope emulsified in the same adjuvant (positive control). These PTPs were purified from mice tumor cell lines that were previously transfected by the Glob-intron-SL8-His construct. After 2 weeks, 50·10⁴ cells from the transfected MCA205 tumor cell line expressing PTPs identical to the purified PTPs, were subcutaneously injected in the right flank of mice. In the left flank of the mice 50·10⁴ wild-type MCA205 tumor cells were similarly inoculated. Inventors' data indicate that PTPs can induce a defect in the tumor growth from the tumor cell line that expresses the PTPs but not from the wild-type (WT) tumor cell lines (FIGS. 3A and 3B), demonstrating the specific anti-tumor effect of the PTPs vaccine.

All those experiments shed light the specific effect of PTPs on tumor growth, and, support the concept that PTPs can be used as vaccine in mice to elicit a specific anti-tumor-T-cell response in prophylactic and therapeutic manners.

### PTPs and Exosomes: A New Cancer-Vaccine

Inventors have recently demonstrated that PTPs are a better source of peptide for the MHCclass I cross presentation pathway than full length protein. Inventors are now reporting that PTPs allows a better cross presentation when stored in vesicles. In fact subcellular fraction that can be released by most of the cells, when smaller than 400 nm, are called microvesicles or exosomes (30-100 nm). To follow this idea, inventors hypothesized that the PTP transfer is mediated by exosomes secreted from the donor cells and internalized by bone marrow dendritic cells (BMDCs). The exosomes from the MCA 205 cell lines were purified according to previous reports. To confirm that the purified materials are exosomes, a FACS analysis was conducted. FIG. 4A reveals the presence of the different surface proteins CD9 and CD81, usual markers of exosomes, confirming that the purified microvesicles from the different cell lines were exosomes. Then, these exosomes were pulsed directly on BMDCs. FIG. 4B (left panel) shows that BMDCs, that have engulfed the MCA 205 tumor-derived exosomes, are capable to activate the CD8+ OT-1 T cells. Since the exosomal purified fraction was from MCA tumor cell lines, a cell line that expressed endogenously the K^{b} molecules, inventors were wondering if the exosomes from this cell lines could have activated directly the CD8+ OT-1 T cells. The derived-MCA exosomes were pulsed directly on the CD8+ OT-1 T cells. No activation of the T cells was seen after addition of the exosomes (FIG. 4B, right panel). In fact when they looked at the expression of the MHC class I K^{b} molecules by FACs analysis, using the anti-K^{b} antibody, they could detect K^{b} molecules, as expected, on the cells surface of the mouse cell lines and in the same time they could not detect K^{b} molecules at the cell surface of the exosomes (FIG. 4C), supporting the fact that MCA exosomes could not by themselves activate CD8+ OT-1 T cells.

So the next step in the vaccin design has been to include in the PTPs-based cancer vaccines, the exosomes of the same tumor cell lines where the PTPs have been purified. For that purpose, inventors have incubated purified PTPs from MCA-205-Glob-intron-SL8 with exosomes from the same tumors for few hours in an adjuvant. Then different groups of 6 mice were vaccinated with different concentration of PTPs, with or without exosomes (15 μg) or with the adjuvant itself (negative control), or with the SL8 epitope emulsified in the same adjuvant (positive control). Inventors' data indicate that the vaccine composed of tumor-derived PTPs with tumor-derived exosomes induce a better defect in the tumor growth (FIG. 4D, cross lines) from the tumor cell line that expresses the SL8 epitope as PTPs (MCA Ova tumor cells) than the vaccines composed only by the tumor-derived PTPs (FIG. 4D, square lines).

### Addition of CD4 Epitope to Improve the PTPs Cancer Vaccine

From above results, inventors have shown that MHC class I peptides incorporated in PTPs and found in exosomes induce a specific anti-tumor response in mice. Nevertheless the main goal of vaccination and especially in cancer treatment is to avoid the relapse of it. And to avoid this relapse, it is necessary to induce a long lasting immunity. It is well established that CD4+ T cells can initiate and extend the life of specific anti-tumor CD8+ T cells and furthermore to induce an accumulation of professional antigen presenting cells (pAPCs) at the tumor sites. This accumulation can be beneficial as PTPs produced by the tumor are a better source for the MHC class I pathway presented by pAPCs than full length proteins. For all those reasons, a vaccine composed of PTPs in combination with the full length protein from the same gene was used. Different groups of 6 mice were vaccinated respectively with PTPs alone, or in combination with the protein Ovalbumin, or with the adjuvant itself (negative control), or with the SL8 epitope emulsified in the same adjuvant (positive control). Inventors data indicate that the vaccine composed of tumor-derived PTPs in combination with the full length protein induce a better defect of the tumor growth (FIG. 5, cross line) from the tumor cell line that expresses the SL8 epitope as PTPs (MCA Ova tumor cells) than the vaccine composed only by the tumor-derived PTPs (FIG. 5, square line) and no effect can be seen of the growth of the WT tumor cell lines, demonstrating the specific anti-tumor effect of the PTPs-full length protein vaccine and supporting the idea that for a better immune response against transformed cells it is preferred to combine peptides activating CD8⁺ and CD4⁺ T cells in the vaccine to induce a better and long-lasting anti-tumor immune response.

### Example 2—Vaccines Against Melanoma

### PTPs-Based Vaccines Against Melanoma:

Inventors have shown in example 1 that PTPs purified from sarcoma cell lines such as MCA205 can be used as vaccine in mice to elicit a specific anti-tumor-T-cell response in prophylactic manner. To expend their idea that PTPs are suitable as anti-cancer vaccine they looked at other types of cancer. For that purpose inventors have purified PTPs from melanoma cell lines such as the murine B16F10 cell line. Then, different groups of 6 mice were vaccinated with different concentration of PTPs, with the adjuvant itself (negative control), or with the SL8 epitope emulsified in the same adjuvant (positive control). These PTPs were purified from mice B16F10 tumor cell lines that were previously transfected with inventors' Glob-intron-SL8-His construct. After 2 weeks, 50·10⁴ cells from the transfected B16F10 tumor cell line, expressing PTPs identical to those which have been purified, were subcutaneous injected in the right flank of mice. In the left flank of the mice 50·10⁴ wild-type B16F10 tumor cells were similarly inoculated.

Inventors' data indicate that PTPs can induce a defect of the tumor growth from the melanoma tumor cell line that expresses the PTPs but not from the wild-type (WT) melanoma-tumor cell lines, demonstrating the specific anti-tumor effect of the PTPs vaccine (FIGS. 7A and 7B).

All those experiments shed light the specific effect of PTPs on any tumor growth subtypes, and, support the idea that PTPs can be used as vaccine in mice to elicit a specific anti-tumor-T-cell response in prophylactic and therapeutic strategies.

### PTPs-Exosomes Based Vaccines Against Melanoma:

Inventors have previously reported that tumor-derived exosomes contain PTPs, and that these exosomes can be associated with PTPs, themselves purified from tumor cell lines, to be used as a cancer vaccine. The exosomes from the B16F10 cell lines expressing the Glob-intron-SL8 construct were purified according to previous reports. The inventors have incubated purified PTPs from B16F10-Glob-intron-SL8 with exosomes from the same tumor for few hours in an adjuvant. Then different groups of 6 mice were vaccinated with different concentration of PTPs, with or without exosomes (15 μg) or with the adjuvant itself (negative control), or with the SL8 epitope emulsified in the same adjuvant (positive control). Inventors' data indicate that the vaccine composed of tumor-derived PTPs with tumor-derived exosomes induce a better defect in the tumor growth (FIG. 8A, square line) from the tumor cell line that expresses the SL8 epitope as PTPs (B16F10 Ova tumor cells) than the vaccines composed only by the tumor-derived exosomes (FIG. 8A, circle black line). From these results the tumor-derived exosomes from melanoma cell lines containing PTPs are stimulating a weak specific immune response, compare to what inventors have seen with the MCA 205-derived exosomes. In fact, the combination of tumor-derived PTPs and tumor-derived exosomes is more potent in inducing a tumor growth defect even if this effect is not enough to induce a complete tumor rejection. Inventors' data indicate also that combination of PTPs and exosomes purified from melanoma cell lines can induced a weak defect in the tumor growth from the tumor cell line that expresses the PTPs but not from the wild-type (WT) tumor cell lines (compare FIGS. 8A and 8B), demonstrating the specific anti-tumor effect of the PTPs-exosomes based vaccine.

### PTPs-Melanosomes Based Vaccines Against Melanoma:

Since exosomes from melanoma cell lines induce a weak defect in tumor growth, the inventors hypothesize that another vesicles released by the melanoma cell lines could have the same effect as the sarcoma exosomes on tumor growth. Melanoma cell lines have the ability to secrete not only exosomes but also melanosomes. In fact melanocytes are specialized in the production of melanin pigment that is stored in organelles called melanosomes (Raposo and Marks, 2007). Melanosomes are a tissue-specific lysosome-related organelle (Raposo and Marks, 2007), classified into two main maturation stages based on morphology and pigmentation level (Watabe, Kushimoto et al., 2005). Immature (stage I and II) melanosomes lack pigment and are located in the central cytoplasm; these are termed “pre-mature melanosomes”. Mature, heavily pigmented melanosomes (stage III and IV) or “mature melanosomes” predominate at distal dendrites, the main site of their secretion.

To follow the idea that the PTP transfer could be mediated by melanosomes secreted from the melanoma donor cells and internalized by bone marrow dendritic cells (BMDCs) as inventors have reported from the sarcoma exosomes, the secreted melanosomes from the B16F10 cell lines were purified according to previous reports. Then these melanosomes were pulsed directly on BMDCs. FIG. 9A shows that BMDCs, that have engulfed the B16F10 tumor-derived melanosomes, are capable to activate the B3Z hybridoma cell lines, which are specific to recognize the MHC class I Kb/SIINFEKL complex at the cell surface. Inventors next tested if inventors could detect the corresponding PTPs inside these secreted melanosomes. For that purpose inventors expressed a construct in B16F10 cells in which the 6×His-tag was inserted next to the SL8 epitope in the intron. Inventors then enriched the PTPs from purified and sonicated secreted stage IV melanosomes using nickel agarose beads and subjected these fractions to LC-MS/MS mass spectrometry analysis. Table 2 shows different peptide fragments carrying, or not, the SL8 epitope. It is worth pointing out that the enrichment step was required in order to yield a sufficient enough concentration of intron-derived PTPs to be detected by MS analysis. This is in line with previous observations showing that despite being an excellent substrate for the endogenous pathway, the PTPs are rare products.

TABLE 2
Mass spectrometry analysis of peptides derived
from melanosomes produced by B16F10 cells
transfected by Glob-inron-SL8 construct. The pep-
tide corresponding to the SIINFEKL peptide
derived from an intron sequence is highlighted.

| | Peptide | Peptide |
|---|---|---|
| Peptide sequence | length, a.a. | origin |
| LEYNYNSHNVYIMADK | 16 | YFP-globin |
| (SEQ06) | | |
| AGYTMVHLTPEEK | 13 | YFP-globin |
| (SEQ12) | | |
| SAMPEGYVQER | 11 | YFP-globin |
| (SEQ02) | | |
| SAVTALWGK | 9 | YFP-globin |
| (SEQ13) | | |
| VNVDEVGGEALGR | 13 | YFP-globin |
| (SEQ01) | | |
| DHMVLLEFVTAAGITLGMDELYK | 23 | YFP-globin |
| (SEQ14) | | |
| FEGDTLVNR | 9 | YFP-globin |
| (SEQ03) | | |
| GEELFTGVVPILVELDGDVNGHK | 23 | YFP-globin |
| (SEQ07) | | |
| AEVKFEGDTLVNRIELK | 17 | YFP-globin |
| (SEQ15) | | |
| GIDFKEDGNILGHK | 14 | YFP-globin |
| (SEQ16) | | |
| TIFFKDDGNYK | 11 | YFP-globin |
| (SEQ17) | | |
| SIINFEK | 7 | Chicken |
| (SEQ05) | | Ovalbumin |
| FSVSGEGEGDATYGK | 15 | YFP-globin |
| (SEQ04) | | |
| SAVTALWGKVNVDEVGGEALGR | 22 | YFP-globin |
| (SEQ18) | | |
| KAGYTMVHLTPEEK | 14 | YFP-globin |
| (SEQ19) | | |
| YQTSLYK | 7 | YFP-globin |
| (SEQ20) | | |
| FSVSGEGEGDATYGKLTLK | 19 | YFP-globin |
| (SEQ21) | | |
| FEGDTLVNRIELK | 13 | YFP-globin |
| (SEQ22) | | |
| AEVKFEGDTLVNR | 13 | YFP-globin |
| (SEQ23) | | |

Moreover, the inventors included in the PTPs-based cancer vaccines as herein described above, the purified secreted stage IV melanosomes from the B16F10 melanocytes. For that purpose, different groups of 6 mice were vaccinated respectively, with 30 μg of secreted stage IV melanosomes, with 16 μg of PTPs purified from B16F10-Glob-intron-SL8 and with the adjuvant itself (negative control). Inventors' data indicate that the vaccine composed of tumor-derived melanosomes of stages IV induce a better defect of the tumor growth from the tumor cell line that expresses the SL8 epitope as PTPs (B16F10 Ova tumor cells) than the vaccines composed only by the tumor-derived PTPs (FIG. 9B). Inventors' data indicate also that melanosomes can induce a defect in the tumor growth from the tumor cell line that expresses the PTPs but not from the wild-type (WT) tumor cell lines (compared FIGS. 9B and 9C), demonstrating the specific anti-tumor effect of the melanosome-containing PTPs based vaccine.

### Discussion

If the main goal of a vaccine is to reduce the chance of transformed cells to escape the host immune system, inventors are demonstrating in this study that i) polypeptides (PTPs) produced earlier by a translation event distinct from the canonical event giving rise to full length proteins can be used as a specific and robust cancer-vaccine, ii) that the combination of such polypeptides and exosomes-carrying similar polypeptides can be an even more powerful combination as a cancer vaccine to trigger a broad T cell repertoire against transformed cells, and that iii) for a long lasting immune response a combination of CD8 and CD4 PTPs is required.

A class I binding synthetic epitope derived from the MAGE-1 protein has been already tested as a single peptide based vaccine in a clinical trial. Then other short peptides directed against different cancer have been used after that. Nevertheless in all of these studies, using single synthetic epitopes as vaccines the expected results were not as good as hoped since they were able to see any beneficial clinical responses in melanoma patients. These results can be explained by the fact that short peptides can bind directly to numerous types of cells and not only to pAPCs that could activate specific CD8+ T cells. Even worse when short peptides bind to MHC class I molecules to non-professional cells, they might induced a tolerance immune response. Also since these peptides are short they might be having any tertiary structure and so being subjected to rapid degradation. For all those reasons, inventors' PTPs-based cancer vaccine seems to be a better strategy than using short peptide. The different reasons are i) inventors' PTPs have been shown to be composed of peptides of different length, longer than 6 amino acids, preferably of at least 7 or 8 amino acids, ii) they have been shown to be the major source of peptides for the endogenous but also the exogenous MHC class I pathway iii) they need to be taken by pAPCs and being properly processed to reach the MHC class I pathway and being presented at the cell surface, iv) they can be composed of MHC class I epitopes but also of MHC class II epitopes. This last reason is very important if the main goal of the vaccine is to induce a long lasting immune response against cancer. In fact, inventors are designing a cancer vaccine to avoid any relapse of any type of cancer. Their vaccine is a therapeutic vaccine where PTPs and exosomes will need to be purified from a patient that has developed already a cancer. The goal of inventor's vaccine is to have a complete tumor rejection and no relapse. For these purposes their vaccine require to induce a quick immune response base on the activation of cytotoxic CD8+ T cells but for a long lasting response the vaccine require also to induce a memory response. In that particular case the memory response will be based on the role of CD4+ T cells. In fact when inventors purified PTPs from WT tumor cell lines (FIG. 2A) they observe a better inhibition of the tumor growth than when they used only the PTPs that come specifically from their engineered model construct where only one MHC class I epitope is used (FIG. 3A). The explanation is that they believe that in the purified PTPs from the WT tumor cell lines not only polypeptides containing MHC class I epitopes are purified but also polypeptides that contain MHC class II epitopes. This observation is supported by the fact that when they mixed PTPs purified from their engineered construct with the full length Ovalbumin, the effect of this combined vaccine is much more potent that when they used only PTPs itself (FIG. 5). CD4⁺ T cells have been shown to be essential for the maintenance of memory CD8+ T cells though the CD40-CD40L interaction between the CD4⁺ T cells and the pAPCs, this again demonstrating the important and specific role of the pAPCs in the success of a vaccination.

According to a series of reports, tumor-derived exosomes have been found to be immunosupressor inducing tumor immune evasion by acting on different pathways, for example by inhibiting the differentiation of DCs or by negatively regulating the NK cells, but they were also reported to have an immunostimulatory effect by inducing a specific tumor-immune response. They have been shown to usually contain tumor antigens and therefore been used as a novel source of tumor antigens for cancer vaccines. From inventors results the tumor-exosomes containing PTPs are stimulating a specific immune response. In fact, the combination of tumor-derived PTPs and tumor-derived exosomes is more potent in inducing a tumor rejection than tumor-derived PTPs themselves (FIG. 4). This result can be explained by the fact that they have succeeded to purify PTPs, produced from their engineered construct, inside the exosomes and that exosomes may also contain MHC class II epitopes coming from the tumor itself.

According to series of reports, melanosomes can be transfer from melanocyte to keratinocytes. In fact series of studies have reported that e.g. the melanosomes are the vesicles that are responsible for the transfer of melanin from the melanocytes to the neighboring keratinocytes. But more importantly for inventors it has been also shown recently that melanoma cells can acquire an MHC class II antigen by intercellular transfer with the help of secreted melanosomes. Here, inventors report that not only MHC class II epitope can be transferred but also MHC class I epitope. In fact inventors have discovered that secreted melanosomes contain PTPs that can be transferred from melanoma cell line to BMDCs with for consequence an activation of specific CD8⁺ T cells. Furthermore, inventors also report that melanosome can be a base for a melanoma cancer vaccine. Inventors showed that injected melanosomes in mice that have been inoculated with melanoma cell lines can induce an important tumor growth defect, supporting the idea that PTPs in combination with melanosomes can be used as a proper melanoma cancer vaccine. Taking into account that an appropriate tumor immune response is dependent on the recruitment and activation of specific CD8 cytotoxic T cells, and the fact that CD4⁺ T cells are necessary to these processes, and the fact that a proper anti-CD8 tumor immune response fail to established long lasting T cell memory in the absence of antitumoral-CD4⁺ T cell, their results show the importance of using melanosomes in a PTPs-based melanoma cancer vaccine, where secreted melanosomes contain MHC class II but also MHC class I epitopes.

### REFERENCES

Apcher, S., C. Daskalogianni and R. Fahraeus (2015). “Pioneer translation products as an alternative source for MHC-I antigenic peptides.” Mol Immunol.
Apcher, S., G. Millot, C. Daskalogianni, A. Scherl, B. Manoury and R. Fahraeus (2013). “Translation of pre-spliced RNAs in the nuclear compartment generates peptides for the MHC class I pathway.” Proc Natl Acad Sci USA 110(44): 17951-17956.
Clayton, A., J. P. Mitchell, J. Court, S. Linnane, M. D. Mason and Z. Tabi (2008). “Human tumor-derived exosomes down-modulate NKG2D expression.” J Immunol 180(11): 7249-7258.
Garin, J., Diez, R., Kieffer, S., Dermine, J. F., Duclos, S., Gagnon, E., Sadoul, R., Rondeau, C. and Desjardins, M. The phagosome proteome: insight into phagosome functions. J Cell Biol 152, 165-80, 2001
Raposo, G. and M. S. Marks (2007). “Melanosomes—dark organelles enlighten endosomal membrane transport.” Nat Rev Mol Cell Biol 8(10): 786-797.
Raposo, G. and M. S. Marks (2007). “Melanosomes—dark organelles enlighten endosomal membrane transport.” Nat Rev Mol Cell Biol 8(10): 786-797.
Thery, C., Boussac, M., Veron, P., Ricciardi-Castagnoli, P., Raposo, G., Garin, J. and Amigorena, S. Proteomic analysis of dendritic cell-derived exosomes: a secreted subcellular compartment distinct from apoptotic vesicles. J Immunol 166, 7309-18., 2001
Thery, C., Regnault, A., Garin, J., Wolfers, J., Zitvogel, L., Ricciardi-Castagnoli, P., Raposo, G. and Amigorena, S. Molecular characterization of dendritic cell-derived exosomes. Selective accumulation of the heat shock protein hsc73. J Cell Biol 147, 599-610, 1999
Thery C., Zitvogel L. and Amigorena S. Exosomes: Composition, Biogenesis and Function. Nature review 2 (2002) 569
Valenti, R., V. Huber, P. Filipazzi, L. Pilla, G. Sovena, A. Villa, A. Corbelli, S. Fais, G. Parmiani and L. Rivoltini (2006). “Human tumor-released microvesicles promote the differentiation of myeloid cells with transforming growth factor-beta-mediated suppressive activity on T lymphocytes.” Cancer Res 66(18): 9290-9298.
Watabe, H., T. Kushimoto, J. C. Valencia and V. J. Hearing (2005). “Isolation of melanosomes.” Curr Protoc Cell Biol Chapter 3: Unit 3 14.
Whiteside, T. L., M. Mandapathil, M. Szczepanski and M. Szajnik (2011). “Mechanisms of tumor escape from the immune system: adenosine-producing Treg, exosomes and tumor-associated TLRs.” Bull Cancer 98(2): E25-31.
Wolfers, J., Lozier, A., Raposo, G., Regnault, A., Thery, C., Masurier, C., Flament, C., Pouzieux, S., Faure, F., Tursz, T., Angevin, E., Amigorena, S. and Zitvogel, L. Tumor-derived exosomes are a source of shared tumor rejection antigens for CTL cross-priming. Nat Med 7, 297-303., 2001
Zitvogel, L., Regnault, A., Lozier, A., Wolfers, J., Flament, C., Tenza, D., Ricciardi-Castagnoli, P., Raposo, G. and Amigorena, S. Eradication of established murine tumors using a novel cell-free vaccine: dendritic cell-derived exosomes. Nat Med 4, 594-600, 1998

1-15. (canceled)

16. A vaccine composition comprising a first Pioneer Translation Product (PTP), said PTP consisting of a peptide having 7 to 50 amino acids, a microvesicle and a pharmaceutically acceptable carrier or excipient.

17. The vaccine composition according to claim 16, wherein the microvesicle comprises a second PTP consisting of a peptide having 7 to 50 amino acids, said second PTP presenting at least one MHC class I epitope and/or at least one MHC class II epitope.

18. The vaccine composition according to claim 16, wherein the composition further comprises the full-length protein corresponding to the first PTP.

19. The vaccine composition according to claim 17, wherein the microvesicles express both the first and at least second PTP, optionally together with at least one third distinct PTP.

20. The vaccine composition according to claim 19, wherein microvesicles are CD8+ T cells activating microvesicles.

21. The vaccine composition according to claim 16, wherein the composition comprises PTPs activating CD4+ T cells and/or CD8+ T cells.

22. The vaccine composition according to claim 16, wherein the vaccine is a cancer vaccine.

23. The vaccine composition according to claim 22, wherein the composition comprises PTPs and microvesicles both derived from the cancerous tumor of the subject to be vaccinated.

24. The vaccine composition according to claim 22, wherein the cancer is a sarcoma or a melanoma.

25. A vaccine composition comprising a nucleic acid sequence encoding a Pioneer Translation Product (PTP) consisting of a peptide having 7 to 50 amino acids and a pharmaceutically acceptable carrier or excipient, wherein the nucleic acid sequence is selected from an intron, a 3′ or 5′ untranslated region (UTR), a LncRNA (Long non coding RNA), a miRNA (microRNA), an intergenic sequence and a combination thereof.

26. A microvesicle comprising a Pioneer Translation Product (PTP) consisting of a peptide having 7 to 50 amino acids expressed from a sequence selected from an intron, a 3′ or 5′ untranslated region (UTR), a LncRNA (Long non coding RNA), a miRNA (microRNA), an intergenic sequence and a combination thereof, said PTP presenting at least one MHC class 1 epitope and/or at least one MHC class II epitope.

27. A method of inducing an immune response in a subject comprising administering a vaccine composition according to claim 16 to said subject.


SEQUENCE LISTING (23 sequences)

SEQ ID NO: 1 - protein, 13 aa, Artificial Sequence
  PTP
VNVDEVGGEALGR

SEQ ID NO: 2 - protein, 11 aa, Artificial Sequence
  PTP
SAMPEGYVQER

SEQ ID NO: 3 - protein, 9 aa, Artificial Sequence
  PTP
FEGDTLVNR

SEQ ID NO: 4 - protein, 15 aa, Artificial Sequence
  PTP
FSVSGEGEGDATYGK

SEQ ID NO: 5 - protein, 7 aa, Artificial Sequence
  PTP
SIINFEK

SEQ ID NO: 6 - protein, 16 aa, Artificial Sequence
  PTP
LEYNYNSHNVYIMADK

SEQ ID NO: 7 - protein, 23 aa, Artificial Sequence
  PTP
GEELFTGVVPILVELDGDVNGHK

SEQ ID NO: 8 - protein, 4 aa, Artificial Sequence
  PTP
SIIN

SEQ ID NO: 9 - DNA, 1161 bp, Gallus gallus
atgggctccatcggtgcagcaagcatggaattttgttttgatgtattcaaggagctcaaagtccaccatgccaatgagaacatcttctactgccccattgccatcatgtcagctctagccatggtatacctgggtgcaaaagacagcaccaggacacaaataaataaggttgttcgctttgataaacttccaggattcggagacagtattgaagctcagtgtggcacatctgtaaacgttcactcttcacttagagacatcctcaaccaaatcaccaaaccaaatgatgtttattcgttcagccttgccagtagactttatgctgaagagagatacccaatcctgccagaatacttgcagtgtgtgaaggaactgtatagaggaggcttggaacctatcaactttcaaacagctgcagatcaagccagagagctcatcaattcctgggtagaaagtcagmcaaatggaattatcagaaatgtccttcagccaagctccgtggattctcaaactgcaakggttctggttaatgccattgtcttcaaaggactgtgggagaaagcatttaaggatgaagacacacaagcaatgcctttcagagtgactgagcaagaaagcaaacctgtgcagatgatgtaccagattggtttatttagagtggcatcaatggcttctgagaaaatgaagatcctggagcttccatttgccagtgggacaatgagcatgttggtgctgttgcctgatgaagtctcaggccttgagcagcttgagagtataatcaactttgaaaaactgactgaatggaccagttctaatgttatggaagagaggaagatcaaagtgtacttccctcgcatgaagatggaggaaaaatacaacctcacatctgtcttaatggctatgggcattactgacgtgtttagctcttcagccaatctgtctggcatctcctcagcagagagcctgaagatatctcaagctgtccatgcagcacatgcagaaatcaatgaagcaggcagagaggtggtagggtcagcagaggctggagtggatgctgcaagcgtctctgaagaatttagggctgaccatccattcctcttctgtatcaagcacatcgcaaccaacgccgttctcttctttggcagatgtgtttccccttaa

SEQ ID NO: 10 - DNA, 1204 bp, Homo sapiens
atggtgcacctgactgatgctgagaaggctgctgtctctggcctgtggggaaaggtgaactccgatgaagttggtggtgaggccctgggcaggttggtatccaggttacaaggcagctcacaagaagttgggtgcttggagacagaggtctgctttccagcaggcactaactttcagtgtcccctgttatgtttccctttttaggctgctggttgtctacccttggacccagaggtactttgatagctttggagacctatcctctgcctctgctatcatgggtaatgccaaagtgaaggcccatggcaagaaagtgataactgcctttaacgagggcctgaatcacttggacagcctcaagggcacctttgccagcctcagtgagctccactgtgacaagctccatgtggatcctgagaacttcagggtgagtctgatgggcacctcgtttccttcccctggctattctgctcaacctttctatcagaaggaaaggggaagcgattctagggagcagtctccatgactgtgtgtggagtgttgacaagagtttggatattttattctctactcagaatcgctgctccctctcactctgttctgtgttgtcatttcctctttctttggtaagcttttaatttccagttgcattttactaaattaattaagctggttatttacttcccatcctgatatcagcttcccctcctcctttcatcccagtccttctctctctcttctctctttctctaatcctttcctttccctcactccatttcttcttctttgatctacttttgtttgtctttttaaatattgccttgtaacttgctcagaggacaaggaagatatgtccctgtttcttctcatagctctcaagaatagtagcataattggcttttatgccagggtgacaggggaagaatatattttacatataaattctgtgtgacataggatcttataataatttgtcagtagtttaaggttgcaaacaaatgtctttgtaaataagcctgcagtatctggtatttttgctctacagttatgttgatggttcttccatcttcccacactcctgggcaatatgatcgtgattgtgctgggccaccacctgggcaaggatttcacccccgctgcacaggctgccttccagaaggtgatggctggagtggccactgccctggctcacaagtaccactaa

SEQ ID NO: 11 - protein, 8 aa, Artificial Sequence
  control peptide
SIINFEKL

SEQ ID NO: 12 - protein, 13 aa, Artificial Sequence
  PTP
AGYTMVHLTPEEK

SEQ ID NO: 13 - protein, 9 aa, Artificial Sequence
  PTP
SAVTALWGK

SEQ ID NO: 14 - protein, 23 aa, Artificial Sequence
  PTP
DHMVLLEFVTAAGITLGMDELYK

SEQ ID NO: 15 - protein, 17 aa, Artificial Sequence
  PTP
AEVKFEGDTLVNRIELK

SEQ ID NO: 16 - protein, 14 aa, Artificial Sequence
  PTP
GIDFKEDGNILGHK

SEQ ID NO: 17 - protein, 11 aa, Artificial Sequence
  PTP
TIFFKDDGNYK

SEQ ID NO: 18 - protein, 22 aa, Artificial Sequence
  PTP
SAVTALWGKVNVDEVGGEALGR

SEQ ID NO: 19 - protein, 14 aa, Artificial Sequence
  PTP
KAGYTMVHLTPEEK

SEQ ID NO: 20 - protein, 7 aa, Artificial Sequence
  PTP
YQTSLYK

SEQ ID NO: 21 - protein, 19 aa, Artificial Sequence
  PTP
FSVSGEGEGDATYGKLTLK

SEQ ID NO: 22 - protein, 13 aa, Artificial Sequence
  PTP
FEGDTLVNRIELK

SEQ ID NO: 23 - protein, 13 aa, Artificial Sequence
  PTP
AEVKFEGDTLVNR